(19) United States
(12) Patent Application Publication
(10) Pub. No.: US 2026/0256535 A1
(43) Pub. Date: Sep. 3, 2026

(54) CELLULAR MANIPULATION VIA MICROROBOTS AND SYSTEMS, METHODS, AND APPARATUS FOR CONTROL THEREOF

(71) Applicants: Max Sokolich, Newark, DE (US); Sambeeta Das Ryan, Newark, DE (US); Sudipta Mallick, Newark, DE (US)

(72) Inventors: Max Sokolich, Newark, DE (US); Sambeeta Das Ryan, Newark, DE (US); Sudipta Mallick, Newark, DE (US)

(73) Assignee: University of Delaware, Newark, DE (US)

(21) Appl. No.: 18/993,084

(22) PCT Filed: Jul. 11, 2023
(86) PCT No.: PCT/US2023/027335

§ 371 Date: Jan. 10, 2025

Related U.S. Application Data
(60) Provisional application No. 63/359,952, filed on Jul. 11, 2022.
(60) Provisional application No. 63/359,960, filed on Jul. 11, 2022.

(51) Int. Cl. A61B 34/00; A61B 34/30; A61B 90/20

(52) U.S. Cl. CPC A61B 34/73; A61B 34/30; A61B 90/20; A61B 2034/303

(57) ABSTRACT

A non-invasive method of manipulating at least one target cell includes treating the at least one target cell with one or more biocompatible magnetic microrobots (MRs); subjecting the one or more MRs to a magnetic oscillation profile via an external magnetic control assembly; and causing the magnetic MRs to move or vibrate in response to the external magnetic control assembly and causing the at least one target cell to move or vibrate in response to motion or vibration of the one or more MRs. The magnetic oscillation profile is configured to cause apoptosis of the at least one target cell. In an embodiment, the one or more MRs deliver a predefined concentration of medication to the at least one target cell. In another embodiment, the one or more MRs rolls toward or adheres to the at least one target cell.

### CROSS-REFERENCE TO RELATED APPLICATIONS

This application claims priority from U.S. Provisional Application Ser. No. 63/359,952, titled “Cell Apoptosis Via Magnetic Field Vibration,” filed Jul. 11, 2022, and U.S. Provisional Application Ser. No. 63/359,960, titled “Electromagnetic Lab on Chip Device for Extracellular Retrieval and Diagnostics,” filed Jul. 11, 2022, the entireties of both of which are incorporated herein by reference.

### STATEMENT REGARDING FEDERALLY SPONSORED RESEARCH

This invention was made with government support under Grant NIH INBRE #P20GM103446 awarded by the National Institute of General Medical Sciences (NIGMS) from the National Institutes of Health (NIH) and the State of Delaware. This invention was also made with support under Grant OIA2020973 from the National Science Foundation (NSF). The government has certain rights in the invention.

### BACKGROUND OF THE INVENTION

Micro-size magnetic particles, or microrobots are untethered devices that are capable of carrying out various tasks at the microscale level. However, prior work on microrobots involve the use of toxic chemicals or light-based actuation mechanisms that are unable penetrate inside the human body. One biocompatible means of driving microrobots relies on magnetic actuation, because magnetic robots can be steered in biological samples without harming cells or tissues. Typically, these microrobots are driven by magnetic gradients which create a magnetic force on the microrobot. In particular, microrobots driven via rotating magnetic fields rather than magnetic field gradients are especially practical for biological applications. Many biological applications involve enclosed environments, such as blood vessels (in which surfaces are abundant), and at air-liquid interfaces, such as the pulmonary system, for example. In this way, the microrobots actuate under the action of a controlled, magnetic field that not only drive or transport them toward a desired trajectory, but also precisely deliver therapeutic payload to the target site. In this way, required concentration of therapeutic molecules can be delivered to the desired site.

Thus, it is of interest to develop improvements in methods and apparatus of controlling microrobots for manipulation of cells, particularly systems, methods and apparatus of controlling microrobots for targeted drug delivery and anticancer therapy.

### SUMMARY OF THE INVENTION

One aspect of the invention is a non-invasive method of manipulating at least one target cell. The method comprises treating the at least one target cell with one or more biocompatible magnetic microrobots (MRs). The method also includes subjecting the one or more MRs to a magnetic oscillation profile via an external magnetic control assembly. The method also comprises causing the magnetic MRs to move or vibrate in response to the external magnetic control assembly and causing the at least one target cell to move or vibrate in response to motion or vibration of the one or more MRs.

Another aspect of the invention is a non-invasive control system of manipulating at least one target cell. The system includes one or more biocompatible magnetic microrobots (MRs); an external magnetic control assembly for subjecting the one or more MRs to a magnetic oscillation profile; and a control device connected to the external magnetic control assembly. The control device is configured to control one or more parameters of the magnetic oscillation profile. The magnetic MRs configured to move or vibrate in response to the external magnetic control assembly and to cause the at least one target cell to move or vibrate in response to motion or vibration of the one or more MRS.

### BRIEF DESCRIPTION OF THE DRAWINGS

The invention is best understood from the following detailed description when read in connection with the accompanying drawings, with like elements having the same reference numerals. When a plurality of similar elements are present, a single reference numeral may be assigned to the plurality of similar elements with a small letter designation referring to specific elements. When referring to the elements collectively or to a non-specific one or more of the elements, the small letter designation may be dropped. This emphasizes that according to common practice, the various features of the drawings are not drawn to scale unless otherwise indicated. On the contrary, the dimensions of the various features may be expanded or reduced for clarity. Included in the drawings are the following figures:

FIG. 1 depicts a non-invasive method of manipulating at least one target cell in accordance with an exemplary embodiment of the invention;

FIG. 2 depicts a schematic of a non-invasive system of manipulating at least one target cell in accordance with an exemplary embodiment of the invention;

FIG. 3 depicts a schematic representation of manipulating target cells via magnetic oscillation or vibration in accordance with an exemplary embodiment of the invention;

FIG. 4 depicts a graph displaying a relationship between frequency and angular displacement in accordance with an exemplary operation of the system of FIG. 2A;

FIGS. 5A-5D depict results of biocompatibility assessments of microrobots in accordance with clinical testing performed with the schematic representation FIG. 3A;

FIG. 6 depicts flow cytometry data showing cell internalization of microrobots in exemplary cell lines in accordance with clinical testing performed with the schematic representation FIG. 3A;

FIG. 7 depicts results showing cell death in accordance with clinical testing performed with the schematic representation of FIG. 3A;

FIG. 8 depicts a schematic representation of manipulating target cells via Doxurobicin-conjugated microrobots (DOX-MRs) in accordance with an exemplary embodiment of the invention;

FIGS. 9A-9D depict results of biocompatibility assessments of microrobots in accordance with clinical testing performed with the schematic representation FIG. 8;

FIG. 10 depicts results of comparative cytocompatibility and cellular uptake study of different surface-functionalized microrobots in accordance with clinical testing performed with the schematic representation FIG. 8;

FIG. 11 depicts microscopic images of microrobots moving toward target cells in response to an external magnetic control assembly in accordance with an exemplary embodiment of the invention;

FIG. 12 depicts images of Fourier transform infrared (FTIR) spectra in accordance with clinical testing performed with the schematic representation FIG. 8;

FIG. 13 depicts images optical and fluorescence images of exemplary cell lines in accordance with clinical testing performed with the schematic representation FIG. 8;

FIG. 14 depicts results of cell death assessment of exemplary targets in accordance with clinical testing performed with the schematic representation FIG. 8;

FIG. 15 is a schematic representation of at least an exemplary external magnetic control assembly in accordance with an exemplary embodiment of the invention;

FIG. 16 is a schematic representation of an exemplary vapor deposition of coating on the microrobots in accordance with an exemplary embodiment of the invention;

FIG. 17 depict scanning electron microscope (SEM) images of the microrobots of FIG. 16;

FIG. 18 depicts a sequence of images showing a rolling microrobot in accordance with an exemplary embodiment of the invention;

FIG. 19 depicts a sequence of images showing another rolling microrobot in accordance with another exemplary embodiment of the invention;

FIG. 20 depicts a graph showing a relationship between frequency and speed of the microrobot of FIGS. 18-19 rolling on solid substrate or air-liquid interface in accordance with another exemplary embodiment of the invention;

FIG. 21 depicts images of showing results of attachment assessments in accordance with clinical testing performed with the schematic representation FIG. 15;

FIGS. 22-23 depict images of showing results of cell viability assessments in accordance with clinical testing performed with the schematic representation FIG. 15;

FIGS. 24A-24B depict images of a 2D traditional coil system for manipulating at least one target in accordance with an exemplary embodiment of the invention;

FIGS. 25A-25B depict images of a 3D Helmholtz-based system for manipulating at least one target in accordance with an exemplary embodiment of the invention;

FIGS. 26A-26B depict images of a Quadrupole Magnetic Tweezer System for manipulating at least one target in accordance with an exemplary embodiment of the invention;

FIGS. 27A-27B depict images of exemplary electronics used for manipulating at least one target in accordance with an exemplary embodiment of the invention;

FIG. 28 depicts an image of an exemplary display in accordance with an exemplary embodiment of the invention;

FIG. 29 depict images of an exemplary trajectory of a self-propelled microrobot in accordance with an exemplary embodiment of the invention;

FIG. 30 depict images of an exemplary trajectory of target cells with ingested microrobots in accordance with an exemplary embodiment of the invention;

FIG. 31 depict images of an exemplary trajectory of an acoustic microrobot in accordance with an exemplary embodiment of the invention; and

FIG. 32 depict images of an exemplary trajectory of polystyrene microspheres in accordance with an exemplary embodiment of the invention.

### DETAILED DESCRIPTION OF THE INVENTION

Aspects of this invention relate to methods and apparatus of controlling microrobots for manipulation of cells, particularly methods and apparatus of controlling microrobots for targeted drug delivery and anticancer therapy.

Although the invention is illustrated and described herein with reference to specific embodiments, the invention is not intended to be limited to the details shown. Rather, various modifications may be made in the details within the scope and range of equivalents of the claims and without departing from the invention.

Additionally, various forms and embodiments of the invention are illustrated in the figures. It will be appreciated that the combination and arrangement of some or all features of any of the embodiments with other embodiments is specifically contemplated herein. Accordingly, this detailed disclosure expressly includes the specific embodiments illustrated herein, combinations and sub-combinations of features of the illustrated embodiments, and variations of the illustrated embodiments.

Many drawbacks of conventional methods and apparatus of controlling microrobots for manipulation of cells are addressed in many respects by devices, methods, and systems in accordance with the invention. While the exemplary embodiments of the invention are described herein with respect to cancer treatment, it will be understood that the invention is not so limited. Suitable applications for methods and apparatus for controlling microrobots of the present invention include, for example diagnostics, targeted drug delivery, cell manipulation, and generally carrying out tasks at the microscale level. More specifically, suitable applications for methods and apparatus for controlling microrobots of the present invention include (i) treatment or procedures requiring microscale maneuvering in blood vessel systems (e.g. for removal of obstructions, etc.), for example; (ii) research or experiments requiring cellular manipulation or delivery (e.g. cell sorting, etc.); (iii) targeted delivery of therapeutic biomolecules for cancer therapy; (iv) and other noninvasive procedures requiring precise control, such as at the microscale level. Other suitable applications will be readily understood by one of ordinary skill in the art from the description herein.

As used herein and throughout the specification, the term “target cell” is intended to encompass the smallest basic functional unit making up tissues and living organisms, and is not limited to a specific type of cell or a specific number or configuration of cells.

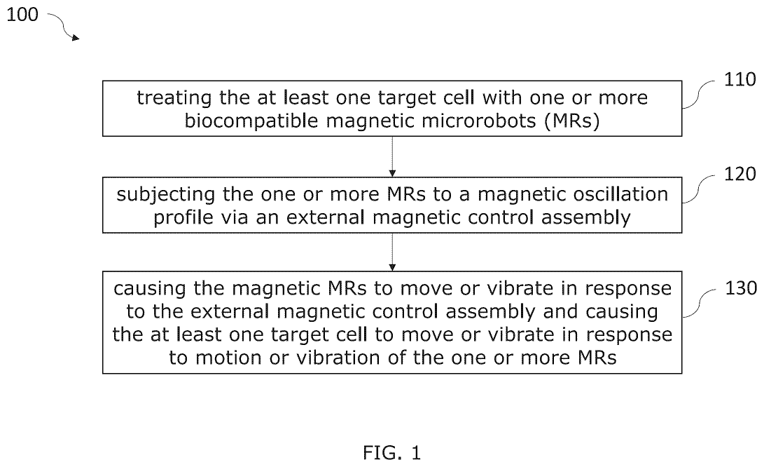

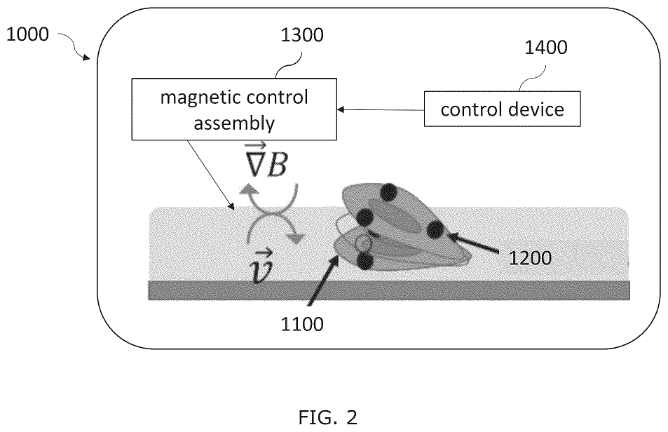

Referring generally to FIGS. 1 and 2A-2C, a non-invasive method of manipulating at least one target or target cell is disclosed. Generally, the method 100 includes a step 110 of treating the at least one target cell 1100 with one or more biocompatible magnetic microrobots (MRs) 1200 and a step 120 of subjecting the one or more MRs 1200 to a magnetic oscillation profile via an external magnetic control assembly 1300. In this way, the method includes a step 130 causing the magnetic MRs 1200 to move or vibrate in response to the external magnetic control assembly 1300 and causing the at least one target cell 1100 to move or vibrate in response to motion or vibration of the one or more MRs 1200. Additional details of the method 100 is discussed below in the context of a non-invasive control system 1000 of manipulating the at least one target cell 1100.

In general, as shown in FIGS. 2A-2C, the non-invasive control system 1000 is adapted for manipulating the at least one target cell 1100. In general, the system 1000 includes one or more biocompatible magnetic microrobots (MRs) 1200, an external magnetic control assembly 1300, and control device 1500 in communication with the external magnetic control assembly 1300. In one example, the biocompatible magnetic microrobots (MRs) 1200 comprises paramagnetic beads having a diameter of 4.7 μm, as designed and manufactured by of Lake Forest, Illinois (Cat. No. FCM-4056-2). However, the invention is not limited to any particular quantity, type, materials, or construction of parts or components. Additionally, or optionally, the external magnetic control assembly 1300 is configured for subjecting the one or more MRs 1200 to a magnetic oscillation profile. To facilitate this, the system 1000 comprises the control device 1500, which is connected to the external magnetic control assembly 1300. In this configuration, the control device 1500 is configured to control one or more parameters of the magnetic oscillation profile. In operation, the magnetic MRs 1200 are configured to move or vibrate in response to the external magnetic control assembly 1300 and the at least one target cell 1100 is configured to move or vibrate in response to motion or vibration of the one or more MRs 1200.

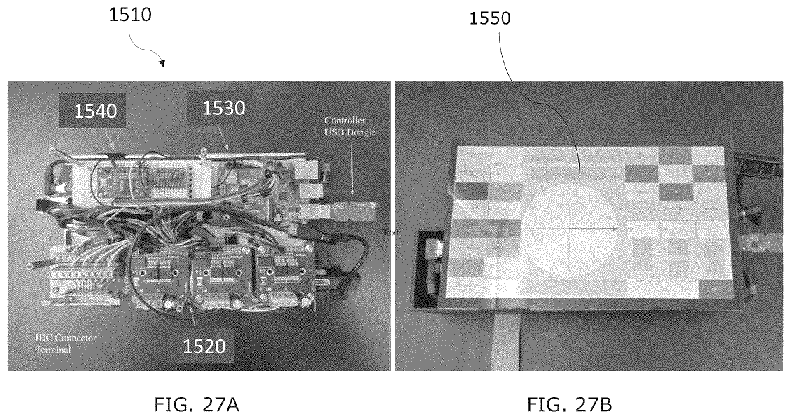

In an exemplary embodiment, control device 1500 comprises a control panel (e.g. a portable control panel) and a processor 1530 (FIG. 27A) coupled to the control panel and the magnetic control assembly. The processor 1530 is configured to transmit an input relating to one or more parameters of the magnetic oscillation profile (as discussed above). In an example, the control device 1500 comprises a portable gaming controller 1510 connected to the control panel via a communication interface, and the portable gaming controller 1510 is configured to provide an input related to a characteristic (e.g. direction) of motion of the one or more MRs 1200.

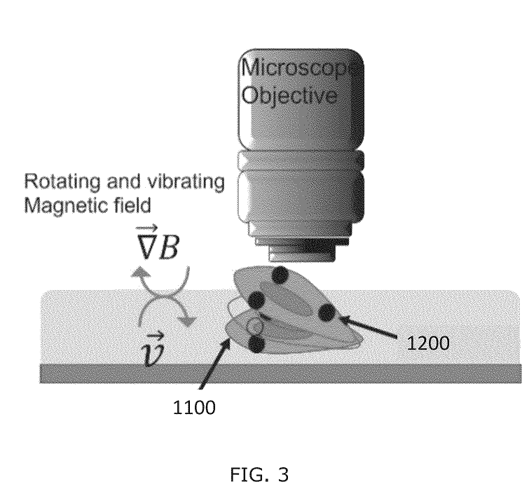

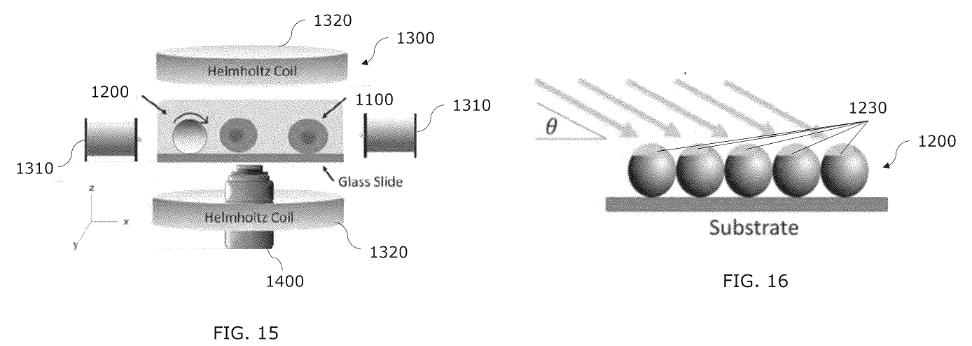

Turning now to FIGS. 3A-3B, 4, 5A-5D, 6, 7, and 15, in an exemplary embodiment, the non-invasive control system 1000 manipulates the at least one target cell 1100 via one or more MRs 1200 configured to cause partial or entire apoptosis (e.g. cell death) of the at least one target cell 1100 via magnetic vibration. To achieve this, as illustrated in FIG. 3A, the magnetic control assembly 1300 is configured to apply magnetic vibration to the one or more MRs 1200 in accordance with a magnetic oscillation profile, and the control device 1500 is configured to control one or more parameters of the magnetic oscillation profile. The one or more parameters include, for example, one or more of a magnetic field orientation and a magnetic field strength provided by the magnetic control assembly 1300. In one non-limiting example, as shown in the set-up of FIG. 3B, the magnetic control assembly 1300 has a plurality of electromagnetic solenoids 1320 arranged in an array along the x and y axes, thereby allowing for magnet fields to be applied in any orientation in the horizontal plane. The magnetic control assembly 1300 also includes a pair of electromagnets 1310 comprising a first electromagnet and a second electromagnet opposite the first electromagnet, as shown in FIG. 15.

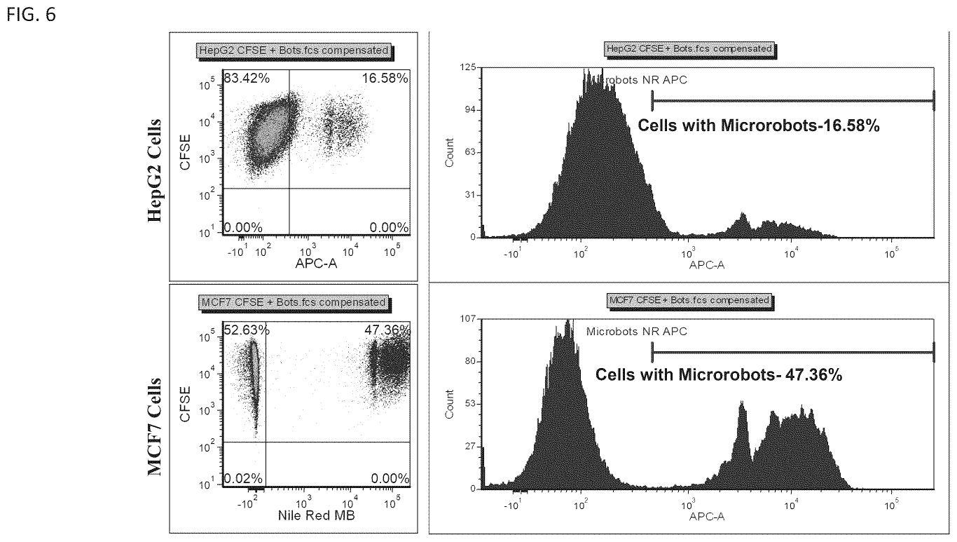

In an exemplary embodiment, FIGS. 3A-3B show that the magnetic control assembly 1300 is installed on a stage of an inverted or upright imaging device, such as a microscope configured to capture real-time images of the at least one target cell 1100 (such as an Axioplan microscope). In this configuration, the at least one target cell 1100 is configured to internalize the one or more MRs 1200. Cellular uptake of the one or more MRs 1200 by the at least one target cell 1100 is facilitated by magnetic actuation of the one or more MRs toward or adjacent the at least one target cell 1100, such that the one or more MRs 1200 are internalized by known internalization pathways, such as endocytosis, in the absence of toxic effects to healthy or non-cancerous cells. The mechanism of cellular uptake can also be due to phagocytosis (e.g. adhesion followed by internalization). For example, the one or more MRs comprising polystyrene beads have displayed a tendency to adhere to the cell membrane of the at least one target cell 1100, followed by internalization. Adhesion may be affected by anionic or cationic charges of the one or more MRs 1200. Studies have shown that the one or more MRs 1200 have a high affinity of with the at least one target cell 1100 (as illustrated in FIG. 6), such as human breast cancer cells (MCF-7).

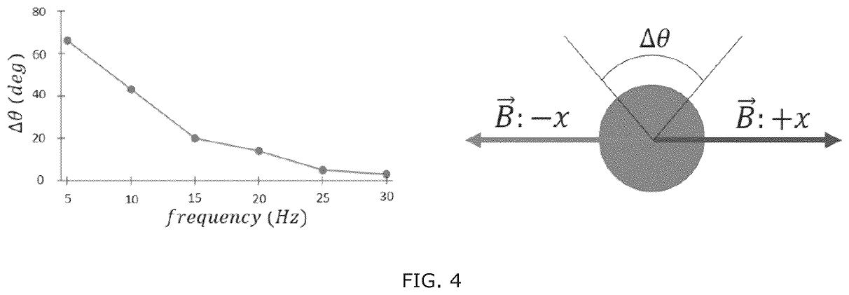

The one or more MRs 1200 are transported or relocated from an origin toward the at least one target cell 1100 via magnetic actuation. As shown in FIG. 4, the increasing frequency results in smaller angular displacement, such that as the frequency at which each electromagnet is pulsed increases, the resulting angular displacement of the one or more magnetic MRs 1200 decreases. In an exemplary embodiment, a frequency of 5 Hz results in the one or more MR 1200 rotating approximately 60 degrees (angular displacement), whereas a frequency of 30 Hz results in relatively smaller angular displacement. Thus, as shown in FIG. 4, the total angular displacement, 40, of the MR 1200 between cycles of magnetic field reversal depends on the applied frequency of the oscillating magnetic field. This is due to the rotational viscous drag on the MR 1200 which prevents it from fully aligning with the applied magnetic field prior to the field reversal. Partial or full apoptosis via magnetic vibration of the at least one target cell 1100 with the internalized one or more 1200s is desired when the at least one target cell 1100 includes a cancer cell. Thus, magnetic oscillation is configured to to disrupt internal structure of the at least one target cell 1100, which leads to subsequent cell death or apoptosis.

When the one or more MRs 1200 are internalized, partial or full apoptosis of the at least one target cell 1100 with the internalized one or more MRs 1200 is facilitated or caused by the magnetic oscillation profile. In particular, the at least one target cell 1100 with internalized MRs 1200 are configured to be aligned and vibrated in accordance with the magnetic oscillation profile. In accordance with an exemplary magnetic oscillation profile, the control device 1500 is configured to apply an oscillating square wave of predefined frequency range for a predetermined duration to the pair of electromagnets 1310. Additionally, or optionally, the magnetic fields are oscillated via rotating magnetic fields (e.g. in the xy plane), as shown in FIG. 3A. In an exemplary embodiment, the one or more parameters of the magnetic oscillation profile includes the predefined frequency range is in a range of 3 Hz to 40 Hz, and the predetermined duration is in a range of 3 to 30 minutes. Additionally, or optionally, the magnetic field strength in a range between 10-30 millitesla (mT). In another embodiment, the magnetic field strength is in a range between 3-7 mT. In still another exemplary embodiment, when the at least one target cell 1100 is a cancerous liver cell, the predefined frequency is 10 Hz, which causes at least partial apoptosis of the at least one target cell 1100 with the internalized one or more MRs 1200.

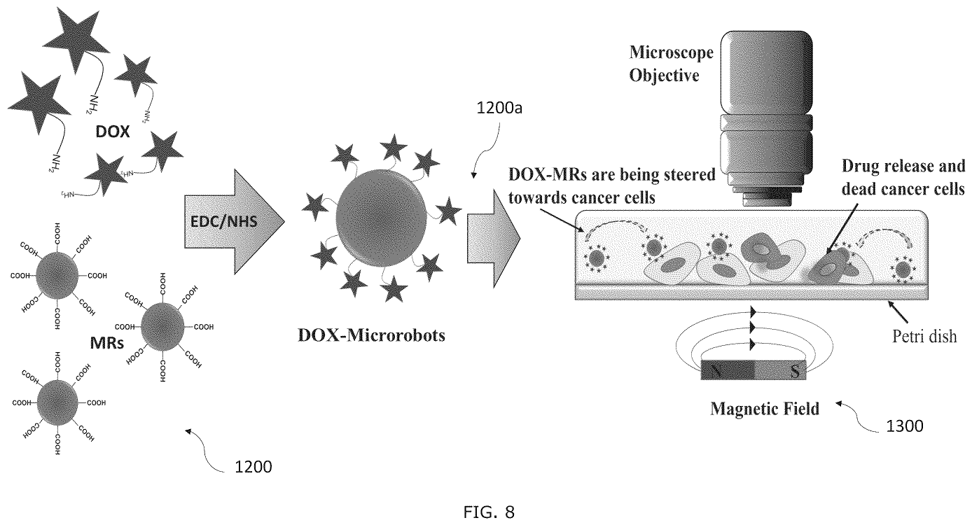

Turning now to FIGS. 8-14, in an exemplary embodiment, the non-invasive control system 1000 manipulates the at least one target cell 1100 via one or more MRs 1200 configured to deliver a predefined concentration of a therapeutic biomolecule to the at least one target cell 1100. In particular, the chemically conjugated MRs 1200a were magnetically steered towards the at least one target cell 1100 using the external magnetic control assembly 1300. To achieve this, as illustrated in FIG. 8, method 100 includes treating the one or more MRs 1200 for conjugation with the therapeutic biomolecule. The methods and systems of the present invention are not limited to a particular therapy or therapeutic biomolecule.

In an exemplary embodiment, the therapeutic biomolecule 1210 comprises Doxorubicin and the one or more MRs 1200 comprises Doxorubicin-conjugated MRs (DOX-MRs) 1200a. As known to one skilled in the art, Doxorubicin (DOX) is an anthracycline that damages DNA or interferes in DNA replication, which eventually, leads to cell death. In this embodiment, the magnetic control assembly 1300 comprises a pair of permanent magnets positionable adjacent the one or more MRs 1200. In this configuration, the magnetic MRs 1200 are configured to selectively move toward the at least one target cell 1100 in response to the motion of the permanent magnets. Thus, method 100 includes a step of delivering a predefined concentration of the therapeutic biomolecule 1210 to the at least one target cell 1100, via the one or more MRs 1200 when the one or more MRs 1200 is internalized by the at least one target cell 1100. In an exemplary embodiment, the at least one target cell 1100 includes a cancer cell. In this way, delivery of the predefined concentration of the therapeutic biomolecule 1210 to the at least one target cell 1100 causes partial or full apoptosis of the at least one target cell 1100. Additionally, or optionally, a decrease in size of the at least one target cell 1100 with the predefined concentration of the therapeutic biomolecule 1210 is observed relative to at least one target cell 1100 without the predefined concentration of the therapeutic biomolecule 1210.

Similar to the set up above, the magnetic control assembly 1300 is configured to apply magnetic field to the one or more MRs 1200 in accordance with the magnetic oscillation profile, and the control device 1500 is configured to control one or more parameters of the magnetic oscillation profile. In one non-limiting example, the magnetic control assembly 1300 has a plurality of electromagnetic solenoids 1320 arranged in an array along the x and y axes, thereby allowing for magnet fields to be applied in any orientation in the horizontal plane. As stated above, the magnetic control assembly 1300 comprises the pair of permanent magnets positionable adjacent the one or more MRs 1200. In an exemplary embodiment, the pair of permanent magnets comprises hand-held permanent magnets that are positionable in closer proximity to the at least one target cell 1100, thereby allowing for application stronger magnetic field gradients. Additionally, or optionally, the one or more parameters include, for example, one or more of a magnetic field orientation and a magnetic field strength provided by the magnetic control assembly 1300. In an exemplary embodiment, the magnetic field strength is in a range between 3-7 mT. In another embodiment, the magnetic field strength is in a range between 10-30 mT. Based on the magnetic field strength, the at least one target cell 1100 are configured to be aligned and the one or more MRs 1200 are configured to steered to move (e.g. roll over, bypass, adhere, etc.) adjacent or toward the at least one target cell 1100 or target site. Upon internalization of the one or more DOX-MRs 1200a, for example, the predefined concentration of DOX was released (e.g. by action of protease enzymes); as a result, partial or full apoptosis of the at least one target cell 1100 or a decrease in relative size of the at least one target cell 1100 is observed.

Turning now to FIGS. 15-23, in an exemplary embodiment, the non-invasive control system 1000 manipulates the at least one target cell 1100 via one or more MRs 1200 configured to move across a solid substrate or an air-liquid interface in response to the external magnetic control assembly 1300. To achieve this, the one or more MRs 1200 is configured to roll toward or adhere to the at least one target cell 1100 in response to magnetic control assembly 1300. Specifically, the rolling MRs 1200 could be maneuvering within blood vessels, such as for removal of obstructions, or in cellular manipulation or delivery, such as cell sorting or biological research experiments as described herein and throughout the specification.

In an example, the one or more MRs 1200 comprises hollow silica spheres and solid silica spheres. As shown in FIG. 16, method 100 includes depositing a full or partial coating 1230 on the one or more MRs 1200 to produce a magnetic moment. In this way, the one or more MRs 1200 are configured to move (e.g. roll) in accordance with the magnetic oscillation profile. In one exemplary embodiment, the one or more MRs 1200 comprise spheres were coated with nickel by e-beam deposition to make them magnetic (as shown in FIG. 16). In particular, the spheres are coated with 100 nm of nickel and the deposition was performed at a 70-degree glancing angle in the case of the silica spheres (as shown in FIG. 16), which generally reduced the surface area that was coated. The magnetic moment of the spheres that have only a partial Ni coating tends to point in a direction tangent to the coated surface, whereas the spheres that are half-coated in Ni produce a magnetic moment that points normal to the surface (visible from the alignment of the one or more MRs 1200 when a magnetic field is applied by the magnetic control assembly 1300). In an exemplary embodiment, the one or more MRs 1200 comprise 45-85 μm diameter hollow silica spheres with a thin TiO₂ coating, as designed and manufactured by Cospheric of Goleta, California. In another exemplary embodiment, the one or more MRs 1200 comprise 20 μm diameter silica spheres with amine functionality, such as those designed and manufactured by Nanocs, Inc. of New York City, New York (Cat. No. Si20u-AM-1).

As shown in FIG. 15, the magnetic control assembly 1300 comprises a plurality of electromagnets 1310 arranged orthogonally along an x-y plane and a pair of Helmholtz coils 1400 positioned opposite of each other for a certain distance, with the one or more MRs positionable therebetween. In operation, rotating magnetic fields are applied in the x-z, or y-z planes, thereby causing the one or more MRs 1200 to move (e.g. magnetic rolling) along an x direction or a y direction, respectively. To apply rotating magnetic fields, discrete sinusoidal signals were sent to each col0 1400l, with a 90 degrees phase difference between the two pairs of orthogonal electromagnets 1310 that corresponded to the desired rotation axis. For example, to apply a rotating field in the x-z plane, the x-axis current was set as Acos(2π f t) and the z-axis as Asin(2π f t), with A the magnitude and f the frequency. In another example, the magnetic field strength is set to 5 mT.

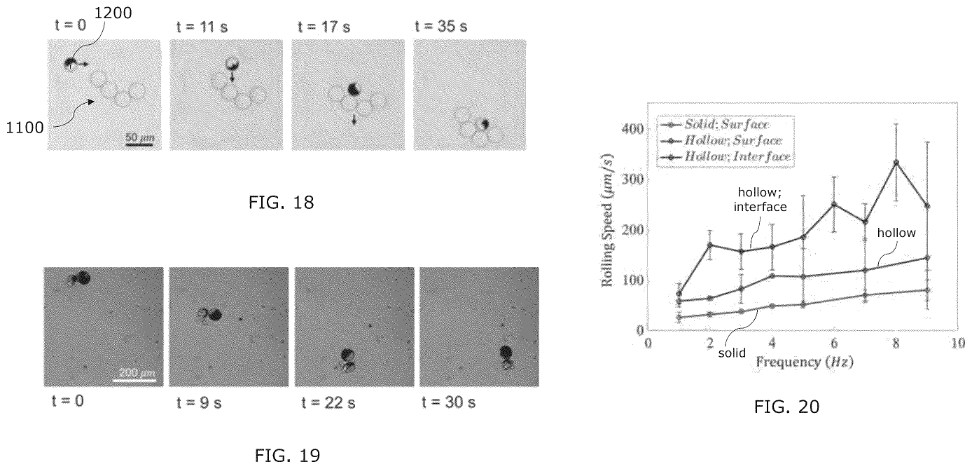

In operation, the one or more MRs 1200 can be moved both on the solid substrate as well as at the air-liquid interface (as shown in FIG. 18). The direction of motion at the air-liquid interface is the same as that on the solid surface for a given rotating field orientations. The speed of the one or more MRs 1200 could be tuned by varying the magnetic field rotation frequency (v). As shown in FIG. 18, the measured speed of the solid silica and hollow MRs 1200 as a function of rotating magnetic field frequency (v), such as up to 10 Hz. Thus, speed of the rolling MRs 1200 increased with frequency approximately linearly, as expected for a rolling object in which the speed is approximately equal to 2πRf, with R being the microrobot radius and f the rotation frequency. On the other hand, due to slip, the actual speed of rolling MRs 1200 in a fluid is reduced. Accordingly, in operation, the one or more MRs 1200 manipulate the at least one target cell 1100 by rolling or pushing the cells or, in some cases, carrying the cells (as shown in FIG. 19).

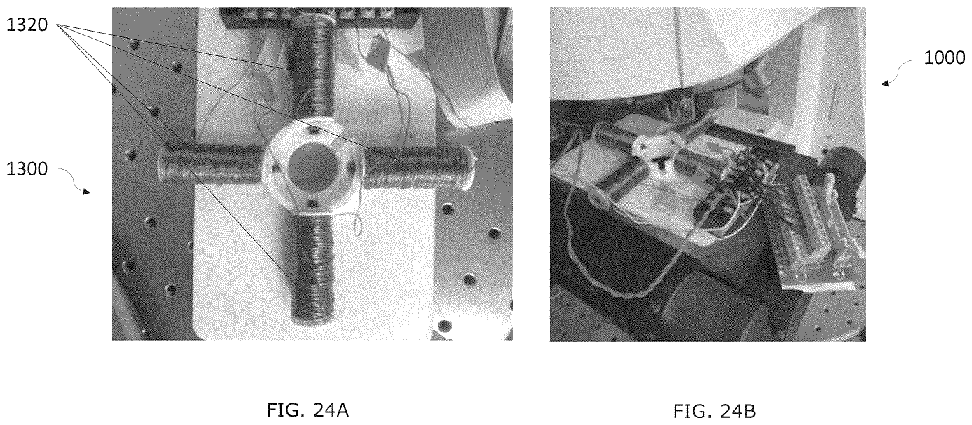

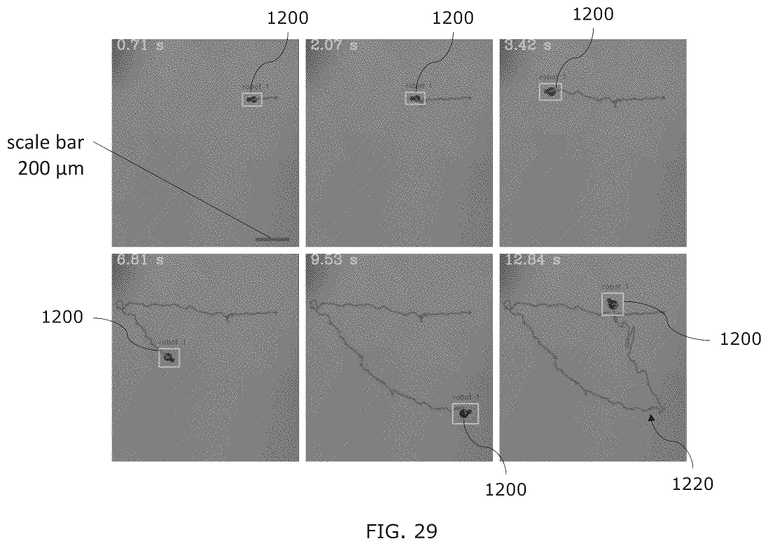

Turning now to FIGS. 24A-24B and FIG. 29, in another exemplary embodiment, the non-invasive control system 1000 includes a magnetic control assembly 1300 comprising a 2D traditional coil system for manipulating at least one target in accordance with an exemplary embodiment of the invention. The components and operation of system 1000 illustrated in FIGS. 24A-24B is similar to those described above. For example, the magnetic control assembly comprises a plurality of electromagnets 1320 arranged in pairs and perpendicular to each other. As shown in FIG. 29, this 2D traditional coil system is useful for manipulating a target, such as self-propelled micro-robots or microrobots (MRs) 1200. Non-limiting examples of such MRS 1200 include electrophoretic or diffusiophoretic magnetic Janus microspheres, or bubble-propelled micro-robots. Namely, FIG. 29 displays an exemplary trajectory 1220 of an MR 1200, such as a 25 μm polystyrene micro-spheres coated in 100 nm Ni and 25 nm Pt, in an air-water interface. In particular, depicted in the images are changes in orientation or direction of the MR 1200 over time (approximately 13 seconds, int his case) and as regulated by the inventive system 1000. Further, fluctuations relative to a perfectly linear trajectory can be due to subtle air currents moving the MR 1200 around.

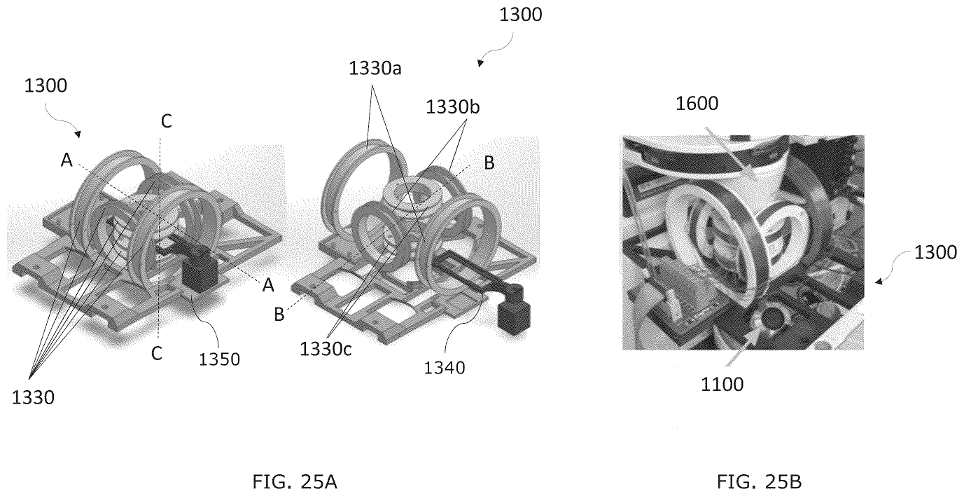

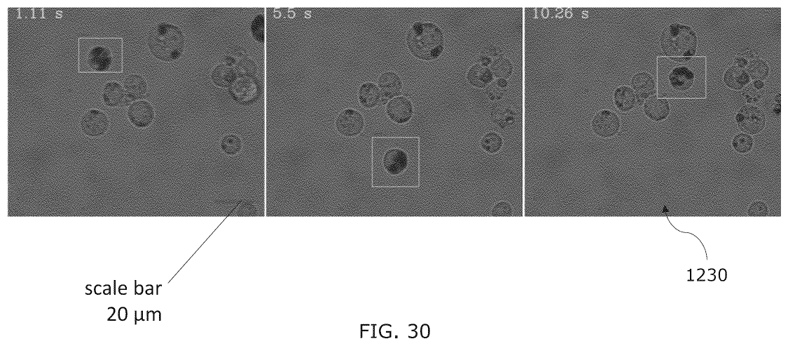

Turning now to FIGS. 25A-25B and FIG. 30, in another exemplary embodiment, the non-invasive control system 1000 includes a magnetic control assembly 1300 comprising a 3D Helmholtz-based system for manipulating at least one target in accordance with an exemplary embodiment of the invention. The components and operation of system 1000 illustrated in FIGS. 25A-25B is similar to those described above. However, there are differences in some respects. For example, in an exemplary embodiment, as shown in FIGS. 25A-25B, the magnetic control assembly 1300 comprises at least one pair of Helmholtz coil rings 1330.

As are known, generally, in the field of microrobotic controls, Helmholtz coil rings, sometimes also referred to as Helmholtz coils (or simply as “rings” hereinafter in shorthand), are configured to produce a homogeneous magnetic field. A typical Helmholtz coil ring pair includes two equal parallel coaxial circular coils connected in series and separated by a distance equal to their common radius. This configuration establishes a known and nearly uniform magnetic field in a region surrounding the center point of the axis between the two coils. As is known in the art, each coil comprises a predetermined number of turns (e.g. 200, 500, etc.) of conductive (e.g. copper, aluminum, etc.) metal wire disposed on a bobbin (e.g. typically plastic). The coils are typically mounted to a base configured to set the proper distance therebetween. Increasing or decreasing the separation between adjacent coil pairs decreases or increases the magnetic field strength, respectively, in accordance with the inverse square law.

In one example, the magnetic control assembly 1300 includes three pairs of rings 1330. The at least one pair of rings 1330 is arranged such that the respective pair of rings are aligned along a common axis. For example, at least one pair of rings 1330 includes a first pair of rings 1330a aligned along the common axis A-A. At least one pair of rings 1330 includes a second pair of rings 1330b aligned along the common axis B-B. At least one pair of rings 1330 includes a third pair of rings 1330c aligned along the common axis C-C. In an exemplary embodiment, the at least one pair of rings 1330 comprise copper wire wrapped around respective outer peripheries of the at least one pair of rings 1330.

As shown in FIG. 25B, the at least one pair of rings 1330 are mounted directly or indirectly on a stage of the microscopic imaging device 1600. In one example, a platform 1350 is configured to be mounted on the stage, and the at least one pair of rings 1330 and a slider arm 1340 are mounted thereon. The slider arm 1340 is configured for translating along a surface of the stage and at least partially within a space defined by the at least one pair of rings 1330 when they are directly or indirectly mounted on the stage. In an exemplary embodiment, a slide or slip is secured on the slider arm 1340, such that the slide or slip moves in response to the motion of the slider arm 1340. The platform 1350, the at least one pair of rings 1330, and the slider arm 1340 comprise plastic material.

Further, the at least one pair of rings 1330 comprise a first pair of rings 1330a spaced apart for a first distance along a first common axis (A-A) that is parallel to the stage. In addition, the first pair of rings 1330a are mounted vertically relative to the stage. Still further, the at least one pair of rings 1330 includes a second pair of rings 1330b spaced apart for a second distance along a second common axis (B-B) that is perpendicular to the first common axis (A-A). In addition, the second pair of rings 1330b is mounted vertically relative to the stage. The second pair of rings 1330b are smaller in size relative to the first pair of rings 1330a. Moreover, the at least one pair of rings 1330 comprise a third pair of rings 1330c spaced apart for a third distance along a third axis (C-C) that is perpendicular to the second axis (B-B) and the first axis (A-A). In addition, the third pair of rings 1330c is mounted horizontally relative to the stage, and the third pair of rings 1330c are smaller in size relative to the first pair of rings 1330a and the second pair of rings 1330b. One skilled in the art would understand that the size of the at least one pair of rings 1330 is not limited to what is illustrated in FIGS. 25A-25B. In one example, a ratio of the first distance over a radius of the first pair of rings 1330a is 1, a ratio of the second distance over a radius of the second pair of rings 1330b is 1.9, and a ratio of the third distance over a radius of the third pair of rings 1330c is 1.6. In an exemplary embodiment, this configuration results in the first pair of rings 1330a configured to apply a magnetic field strength of 2 mT, the second pair of rings 1330b configured to apply a magnetic field strength of 2 mT, and the third pair of rings 1330c is configured to apply a magnetic field strength of 4 mT.

Turning to FIG. 30, the system 1000 comprising the 3D Helmholtz system is utilized to control various types of magnetic rolling MRs 1200. In one example, MR 1200 includes rolling micro-spheres coated with a hemisphere of nickel (via electron beam deposition). In other examples, the 3D Helmholtz system is used to facilitate control of the at least one target cell 1100 with ingested magnetic MRs 1200 (as shown in FIG. 30). Still further, the system 1000 comprising the 3D Helmholtz system is suitable for actuating magnetic helical micro-robotic swimmers 1200. An exemplary trajectory 1230 of the at least one target cell 1100 with ingested MR 1200, such a synthetically engineered Chinese hamster ovarian cell with ingested magnetic microrobots, is illustrated in FIG. 30. In this example, MR 1200 includes 4.8 μm silica micro-beads coated with 100 nm of Nickel.

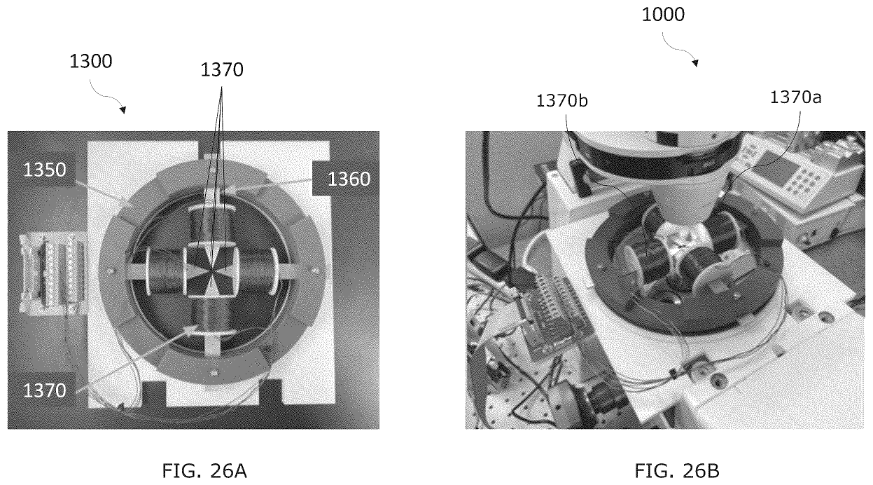

Turning now to FIGS. 26A-26B and FIG. 32, in another exemplary embodiment, the non-invasive control system 1000 includes a magnetic control assembly 1300 comprising a Quadrupole Magnetic Tweezer System for manipulating at least one target in accordance with an exemplary embodiment of the invention. The components and operation of system 1000 illustrated in FIGS. 26A-26B is similar to those described above. However, there are differences in some respects. For example, in an exemplary embodiment and as shown in FIG. 26A, the magnetic control assembly 1300 comprises a yoke 1350 having a plurality of magnetic poles 1360. Each one of the plurality of magnetic poles 1360 has an electromagnetic coil 1370 and a tip 1370 that is spaced apart from each other and oriented radially inward. Each of the tips 1370 of the plurality of magnetic poles 1360 is configured to generate a magnetic field gradient of 26 T/m. Additionally, or optionally, the plurality of magnetic poles 1360 comprises ferromagnetic alloy and the electromagnetic coil 1370 comprises enameled copper wire.

In this configuration, the magnetic oscillation profile comprises modulation of a current applied to the magnetic control assembly 1300. For example, when the magnetic control assembly 1300 comprises a first electromagnet coil 1370a and a second electromagnet coil 1370b (FIG. 26B) arranged opposite the first electromagnet coil 1370a, a first polarity signal is applied to the first electromagnet coil 1370a and a second and opposite polarity signal is simultaneously applied to the second electromagnet coil 1370b, thereby applying a uniform magnetic field strength to the MRs 1200. To facilitate this operation of the magnetic control assembly 1300 in accordance with the magnetic oscillation profile, the control device 1500 includes a magnetic tweezer configured to create magnetic gradients for moving or vibrating the MRs 1200. Additionally, or optionally, the magnetic oscillation profile comprises alignment of a magnetic moment with the magnetic field strength via the magnetic tweezer.

As shown in FIG. 32, system 100 comprising a magnetic tweezers system utilizes high magnetic field gradients to direct magnetic micro-beads 1200. In an exemplary embodiment, this configuration is suitable in cases where self-propulsion by MR 1200 is not feasible or when high forces are required. Notably, FIG. 32 shows exemplary trajectories 1240 of two MRs 1200, such as 25 μm polystyrene microspheres coated in Nickel, moving through water on a microscope slide as regulated by system 1000 comprising magnetic tweezers.

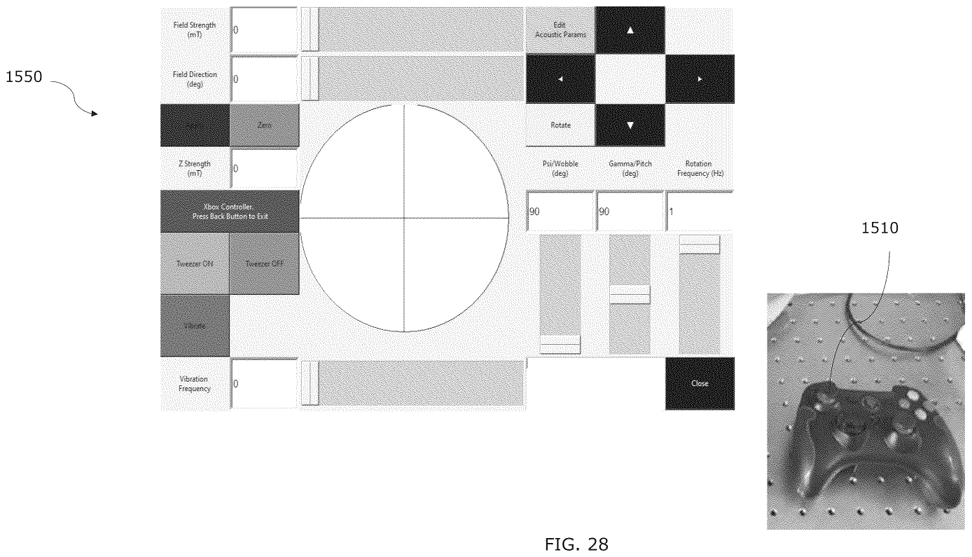

Referring now to FIGS. 27A-27B and 28 depict images of exemplary electronics and display used for manipulating at least one target in accordance with an exemplary embodiment of the invention. FIG. 27A is a rear view with a cover or housing removed to shown the internal components. FIG. 27B is a front view of the control device 1500, which comprises an exemplary user interface 1550. In an exemplary embodiment, the control device 1500 comprises drivers 1520 configured to vary a current and a polarity applied to the magnetic control assembly 1300 by an external power supply. In one example, the external power supply comprises a plurality of rechargeable batteries.

Additionally, or optionally, the control device 1500 comprises a signal generator module 1540 connected to a piezoelectric transducer. In this example, the signal generator module 1540 is configured to produce an electrical sine wave at a frequency and the piezoelectric transducer converts the electrical sine wave into pressure waves at the frequency. In this way, when MRs 1200 comprises acoustic MRs, the pressure waves travel toward the MRs 1200 and are configured to generate a vibration that produces an acoustic streaming force for moving or propelling the MRs.

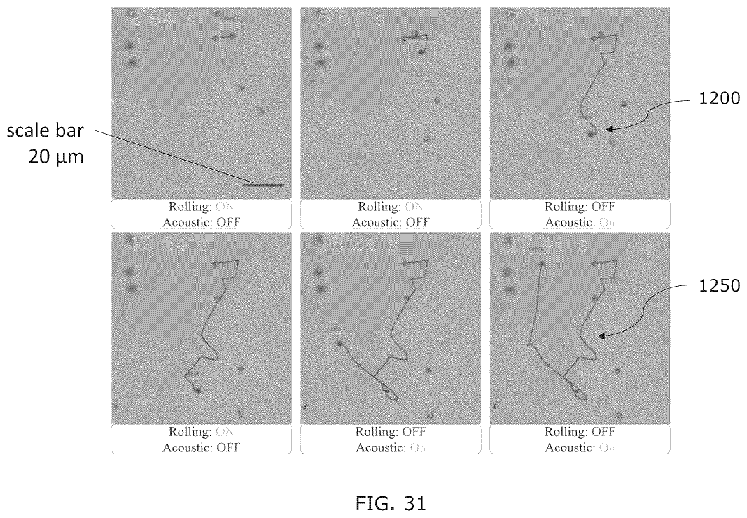

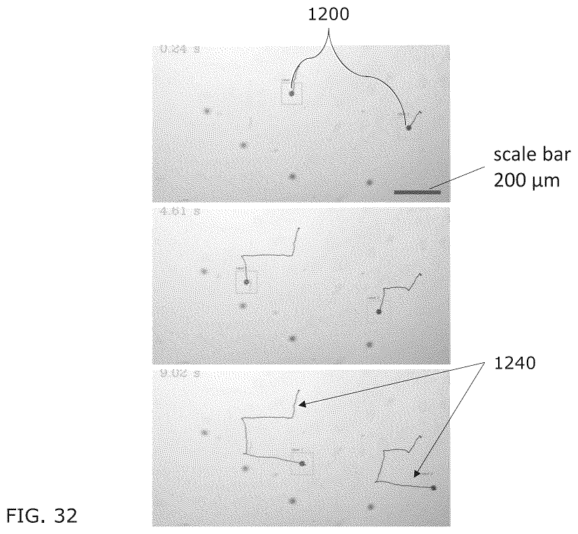

FIG. 31 depict images of an exemplary trajectory 1250 of an acoustic microrobot 1200. As illustrated, one of the MRs 1200 includes a trapped air-bubble for actuation in response to acoustic or pressure waves applied thereto. In this embodiment, the signal generator module 1540 in combination with application of rotating magnetic fields steer or move the acoustic micro-swimmers 1200. To Facilitate this, a “left” joystick of the gaming controller 1510 is used to orient the micro swimmers 1200 while toggling the acoustic signal applied thereto. The system 1000 also allows for rolling of the acoustic micro-swimmers 1200 with the acoustic signal on or off, thereby providing more refined and flexible control options.

Thus, the software and electronic circuitry of system 1000 is suitable for use with multiple electromagnetic setups, such as those described above (e.g. 2D traditional coils system, 3D Helmholtz-based system, and quadrupole magnetic tweezer system, each of which provides micro-robots 1200 with different actuation mechanisms. In this way, the system 1000 is versatile and does not require a complete system redesign when using different micro-robots 1200 or for use with different applications or experiments. Additionally, or optionally, system 1000 is portable and inexpensive, in that the system 1000 is transferable to different microscopes and imaging systems and to non-lab settings, such as for medical applications. Control capabilities also desirably include acoustic actuation, thereby allowing for both acoustic driven and electromagnetic driven microrobotic motility control. In this way, switching seamlessly between magnetic rolling and acoustic actuation of a single microrobot 1200 is provided. In particular, system 1000 comprising any or a combination of the electromagnetic set ups discussed above can be used simultaneously with acoustic actuation via a transducer.

### EXAMPLES

The co-inventors assessed feasibility and functionality of the components of the devices, methods, and systems as disclosed herein, as well as verified any updates or improvements made. The prototype devices, methods, and systems were subjected to various clinical tests as detailed herein.

### Magnetic Microrobots for Liver Cancer Cell Manipulation and Treatment

Microrobots, in particular, have recently been studied for their ability to navigate difficult-to-reach regions in the human body to deliver therapeutics for microscopically localized interventions. However, the control of individual and swarms of microrobots to precisely target localized cellular regions is challenging. Thus, the objective of this study is to provide a method of cancer therapy which utilizes physical manipulation of cancer cells with magnetic microrobots which leads to cell death due to apoptosis.

In a living organism, every single activity is a function of all the biochemical reactions that happen at a cellular level. These biochemical reactions are often a collection of all the synchronized processes that are carried out by subcellular organelles inside the cells. Changing these highly specialized units can corrupt specific or multiple biological signals which often lead to disease onset. Cancer is the simplest example of a dysfunctional mechanism inside a cell that causes uncontrolled growth of the cells. Birth defects are another example where abnormalities in a single cell lead to impaired development of the fetus. Furthermore, correlations between abrupt changes in intracellular electric current across the cell and cardiac arrhythmia are also evident from research data. Likewise, dysfunctional cellular mechanisms are the apparent cause of neurodegenerative diseases and diabetes. Therefore, single cell manipulation at the subcellular and sub-organelle level provides better understanding of diseases and subsequently, developing therapeutic approaches for them.

### Example 1

4.7 μm diameter magnetic microrobots were manipulated for vibration of cancer cells (to trigger cell death). These microrobots were internalized by cancer cells through endocytosis without any toxic effects. Cancer cells with internalized microrobots were then aligned in the direction of magnetic fields and oscillated via fast rotating magnetic fields in the xy plane throughout the experiments (as shown in FIG. 3A). Magnetic oscillation of varying dosages was applied to disrupt internal structure of cancer cells which leads to subsequent cell death.

### Experimental Set-Up

A compact magnetic controller is installed on the stage of an inverted or upright microscope that helps in capturing real time images of cells. These magnetic controllers can be custom designed using 3D printing to fit any microscope (optical or fluorescence microscope) based on experimental design. Moreover, wireless joystick controllers provide precise control over microrobots that are being driven inside the cell. Experiments were carried out using an Axioplan microscope with a custom-built magnetic control assembly or system (as shown in FIG. 3B). The strength and direction of the fields are controlled using custom MATLAB code.

### Materials and Methods

The microrobots consisted of paramagnetic beads (diameter=4.7 μm), as designed and manufactured by Spherotech of Lake Forest, Illinois (Cat. No. FCM-4056-2). However, the invention is not limited to any particular quantity, type, materials, or construction of parts or components.

### Magnetic Experimental Setup of Microrobots

Experiments were conducted on an Axioplan 2 upright microscope using an Axiovert 503 mono camera and an Axiovert 200 inverted microscope with an Amscope MU903-65 camera. Experiments on the Axioplan microscope utilized a custom-built magnetic control system which applied magnetic field strengths at the sample from 3-7 mT. The magnetic system consists of four electromagnetic solenoids containing soft iron cores which are arranged in an array along the x and y axes, allowing for magnet fields to be applied in any orientation in the horizontal plane. The strength and direction of the fields are controlled using custom MATLAB code. The MATLAB program utilizes the Data Acquisition Toolbox and a Wireless gaming controller. The program connects to the controller through Bluetooth® and each button can be mapped to a certain digital output on a National Instruments DAQ USB 6351, and thus a specific electromagnet. Experiments performed on the Axiovert microscope utilized hand-held permanent cylindrical magnets that produced magnetic field strengths at the sample of approximately 10-30 mT. Paramagnetic beads used in the experiments were fluorescent and had a diameter of 4.7 μm. The fluorescent microspheres were illuminated using X-Cite mini+ LED source, as manufactured and designed by Excelitas Technologies Corp. of Salem, Massachusetts.

### Cell Culture and Maintenance

Human breast cancer cells (MCF-7) and Hepatocellular Carcinoma cells (HepG2) cells were provided by Richard West (Associate Scientist at Flow Cytometry Core Facility). Cells were cultured in Dulbecco's Modified Essential Medium (DMEM) and Ham's F-12 (Gibco, BenchStable, USA) media with 5% CO2 and maintained at 37° C. in an incubator. All experiments were performed after third passage of cells.

### Assessment of Cytotoxicity and Cellular Uptake of Microrobots

Cytotoxicity of MRs was evaluated in MCF-7 and HepG2 cells. Cells were seeded (2×10⁴ cells/well) in a clear flat bottom 24-well plate (Corning™ Costar™ USA) and incubated in 1:1 mixture of Dulbecco's Modified Essential Medium (DMEM) and Ham's F-12 (Gibco, BenchStable, USA) media with 5% CO2 at 37° C. for 24 hrs. Then, cells were treated with MRs (4.7 μM size, Yellow, 1 mg/mL) and incubated for 24 hrs. Cells were then imaged under optical microscope to check cell morphology. Flow cytometry was also performed to quantify exact percentage of cell death after propidium iodide (PI) staining. Cellular internalization of microrobots were assessed by flow cytometry. Samples for cellular uptake were prepared as described above in both MCF-7 and HepG2 cells. 1×10⁵ cells/well were seeded in a 6-well plate and incubated in DMEM/F-12 with 5% CO2 at 37° C. for 24 hrs. Then, 20 μL of 1% w/v MRs solution was added to the each well containing 2 mL media. After 24 hrs, cells were washed and stained with CFDA-SE for 15 mins and analyzed using a flow cytometer, such as the BD FACSAria™ IIu flow cytometer, as manufactured and designed by BD Biosciences of Franklin Lakes, New Jersey.

### Cancer Cell Killing Using Magnetic Oscillation

HepG2 and MCF-7 cells were treated with microrobots (100 μg/mL) n a 6-well plate (Costar™ Corning™ USA) and incubated in DMEM/F-12 (Gibco, BenchStable, USA) media with 5% CO2 at 37° C. for 24 hrs. After 24 hrs, cells were washed and trypsinized to detach cells from the culture dish. Then, single cells with internalized particles were subjected to magnetic oscillation (frequency range: 5-40 Hz for both HepG2 cells and MCF- 7 cells, respectively) with different frequency in xy-plane for 3minutes

### Control and Transport of Microrobots

Experiments were carried out using the magnetic system set up (as shown in FIGS. 3A-3B). Magnetic microrobots were transported from one assigned place to another via rotating magnetic fields. As shown in FIG. 4, the graph indicates how increasing frequency results in smaller angular displacement. As the frequency at which each electromagnet is pulsed increases, the resulting angular displacement of the magnetic MR decreases. For example, a frequency of 5 Hz results in the MR rotating approximately 60 degrees whereas a frequency of 30 Hz results in relatively smaller angular displacement.

### Cyto-Compatibility of Microrobots

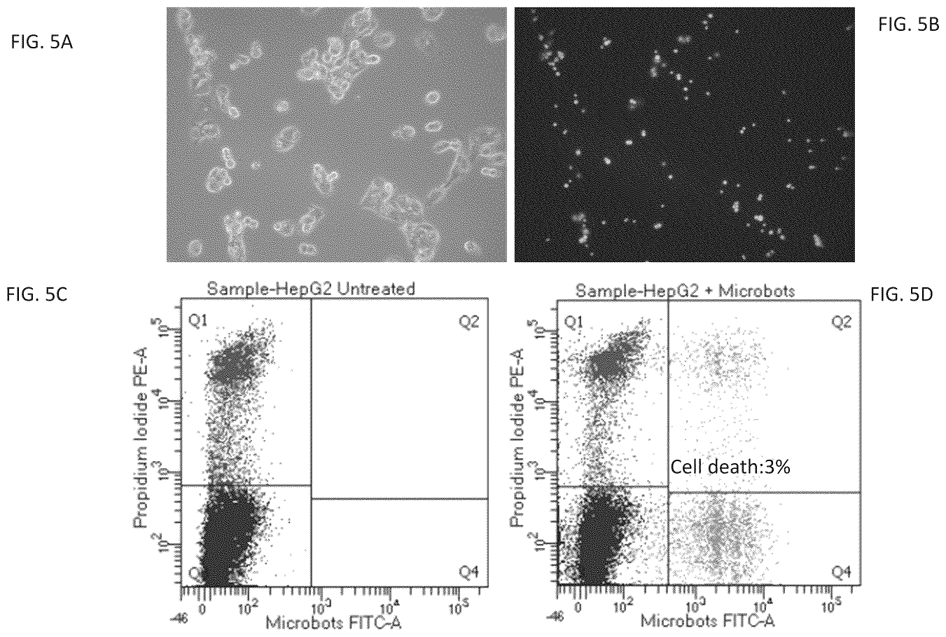

The paramagnetic beads are made of nontoxic polystyrene. Cells were incubated with the microrobots for 24 hrs, and changes in cell morphology was monitored using an optical microscope. The cell monolayer was intact without any toxic effects (as shown in FIGS. 5A-5B). Quantitative analysis was also consistent with the data showing negligible cell death of 3% (FIG. 5D) when compared with untreated cells (as shown in FIG. 5C).

### Cell-internalization of Microrobots

The microrobots were completely internalized by the cells after 24 hrs. There were multiple microrobots observed inside the cells showing high affinity of these polystyrene beads towards the cells. However, higher cell internalization was observed in MCF-7 cells than HepG2 cells, as confirmed by fluorescence microscopy. Flow cytometry data was also consistent with fluorescence microscopic images. Cell internalization in MCF-7 cells was 3-fold higher as compared to HepG2 cells with a broad range of intensities demonstrating inequal distribution of microrobots insides the cells. This difference in cell internalization was presumably due to the cluster forming growth pattern of HepG2 cells (as shown in FIG. 6).

### Cell Transport and Manipulation

Intracellular manipulation using microrobots provides an opportunity to potentially alter or interfere with the cell's biological functionality. Applying intracellular manipulation approach to disrupt the internal structure of cancer cells via magnetic oscillation of the microrobots which ultimately leads to cell death was hypothesized. Once the microrobots are internalized, an oscillating square wave of predefined frequency to opposite facing electromagnets was applied. As a result, the electromagnets rapidly changed from an on state to an off state asynchronously, thereby resulting in a magnetic oscillation of the microrobots. Alternating the direction of the magnetic field from north to south results in an oscillating rotation of a paramagnetic microrobot.

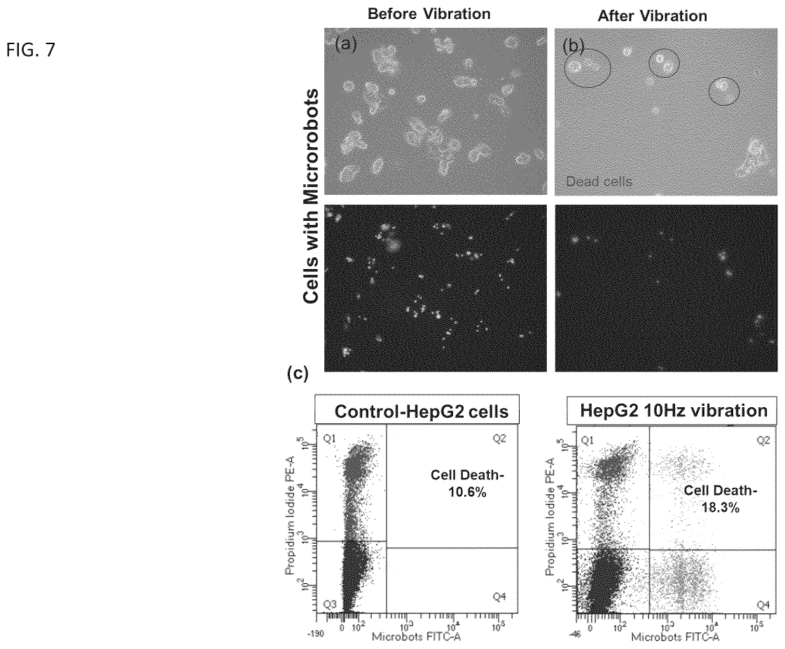

As shown in FIG. 6, the total angular displacement, 40, of the microrobot between these cycles of field reversal depended on the applied frequency of the oscillating magnetic field. This is due to the rotational viscous drag on the microrobot which prevents it from fully aligning with the applied magnetic field prior to the field reversal. Results from this experiment showed that the HepG 2 cells were responsive to oscillation and were dead after 24 hrs (FIG. 7), whereas MCF-7 cells were unresponsive to the applied oscillation even at higher frequencies (closer to 40 Hz), presumably owing to their highly specialized characteristics that make them more robust. HepG2 cells were comparatively more responsive at lower frequencies (around 5-10 Hz) and increased cell death was observed just after one application (as shown in FIG. 7, comparing cell death at 10.6% pre-oscillation and 18.3% at post-oscillation). Moreover, cells with multiple number of microrobots were dead due to the stronger oscillational force collectively generated by all microrobots. This indicates a potential relationship between internal disruption and biochemical signaling. The mechanism of internal disruption and cell death may be caused by shear stress that is generated in the cytoplasm due to the magnetic oscillation (i.e. mechanical stress-induced cancer cell death using microparticles).

Thus, low frequency mechanical vibration at 10 Hz promotes cell death in liver cancer cells, while breast cancer cells were unaffected, suggesting that low frequency mechanical vibration has the potential for selective cancer cell death. In addition, since an increase in temperature is unlikely to be elicited t such a low frequency, the cell death is supposed to be due to apoptosis, which is preferable to reduce the possibility of inflammation and immune cell activation. The risk of damage to nearby cells due to necrosis through heat diffusion is also reduced.

Precise controllability over our magnetic system also provides the ability to accurately navigate the robots to the complex locations in a cellular system. The mechanisms underlying the effects of mechanical vibration on apoptosis are largely unknown, but the results of this study indicate that microrobots can be an effective and non-invasive approach to analyze and manipulate singles cells with improved accuracy and repeatability. Additionally, microrobots can be effectively targeted to the desired location and serve as delivery systems for various biomedical applications. Other suitable applications of magnetic oscillation-based micromanipulation of singles cells includes improvement in understanding of cell behavior and related biochemical outcomes.

### Example 2

### Doxorubicin-Loaded Microrobots (MRs) for Targeted Drug Delivery and Anticancer Therapy

Cancer is the second leading cause of deaths worldwide after cardiovascular diseases according to the World Health Organization (WHO). Although significant progress has been achieved in the development of anticancer therapies and post-treatment management, drug resistance and highly aggressive cancers are still major challenges to overcome. Nevertheless, delivering therapeutic concentration of anticancer agents to target sites of an advanced cancer patient without compromising quality of life is difficult. Apart from conventional cancer therapy, other strategies like nanomedicine, targeted-and immune therapy are currently approved treatment options for various cancers.

Targeted therapy is mainly focused on delivering maximum concentration of therapeutic molecules to the cancer site without affecting normal cells. While different strategies like hormone therapy, signal transduction inhibitors, angiogenesis inhibitors directly interfere or block cellular and molecular mechanisms of cancer growth and spread, monoclonal antibody and cytotoxic drugs aim to kill cancer cells. Besides that, cancer cell surface receptors and glucose molecules are either directly conjugated to anticancer drugs or drug delivery systems to maximize drug accumulation in tumor site.

Micro-sized robots (microrobots; MRs) are highly desirable because of their size that can be visualized under a microscope setup compared to nano-size robots. These MRs can be driven by various chemical (e.g. H₂O₂, H₂O, NaBH₄, etc.) and external energy field (e.g. magnetic field, ultraviolent (UV), ultrasound) through narrow and confined space with precise control. Magnetic MRs are of great interest due to their ability to move under the influence of a magnetic field and control their direction and speed wirelessly. Nevertheless, magnetically controlled MRs can be steered to different inaccessible parts of the body without any invasive procedure. Moreover, diverse structural and surface properties of magnetic MRs offer potential applications in the field of delivery systems, tracking and single cell manipulation.

Prior work on MRs include drugs incorporated with MRs through physical adsorption, electrostatic interaction, and covalent linkage. Different structures, such as spherical, rod-shaped, and helical MRs, were optimized to maximize drug loading and propulsion efficiency. Several groups have reported helical MRs for Doxorubicin (DOX) delivery and release by UV or near infrared (NIR) laser light irradiation. Incorporation of magnetic particles provides MRs with three different (rolling, tumbling, and spinning) motions for propulsions of MRs in different environments. Another study has reported tri-magnetic bead MRs prepared by surface-functionalized biotin-streptavidin interaction for anticancer therapy. However, these strategies involved complex fabrication mechanism of MRs with difficult experimental set of magnetic field controller.

Thus, this example was intended to provide a simple, yet effective strategy to prepared DOX-loaded MRs (DOX-MRs). Specifically, Doxorubicin (DOX) was chemically conjugated with MRs (DOX-MRs) and magnetically steered towards cancer cells using an external magnetic controller. The MRs were used to deliver anticancer drugs to cancer cells. As shown in FIG. 8, anticancer effect of DOX-loaded spherical magnetic beads in different cancer cells (HepG2, MCF-7, Prostrate and ovarian cancer cells) were studied based on subsequent, cell death in the above-mentioned cell lines. Cytocompatibility studies showed that MRs were well-tolerated and internalized by cancer cells. Time-lapsed video showed that cells were shrunk and eventually dead when MRs were internalized by cells. The tryphan blue assay also confirmed cell death in spheroids of HepG2 cells when treated with DOX-MRs.

### Materials and Methods

Paramagnetic beads (diameter—4.7 um) were purchased from Spherotech Cat.

No. FCM-4056-2. Doxorubicin HCL was purchased from Sigma-Aldrich. However, the invention is not limited to any particular quantity, type, materials, or construction of parts or components.

### Preparation of DOX-MRs

Doxorubicin (DOX) was covalently conjugated to magnetic beads by EDC/NHS coupling. 10 mg of magnetic beads were ultrasonically dispersed in 2 mL MilliQ water with 5-times excess EDC and NHS and stirred for 2 hrs. Doxorubicin (DOX conc=1 mg mL-1) was added dropwise to above solution and stirred for 4 hr at room temperature, protected from light. DOX-MRs were collected using centrifugation (12,000 RPM, 30 min, 24° C.). The supernatant was collected separately; solid DOX-MRs were washed with MilliQ water (2 mL×3) until the supernatant became clear, indicating the absence of free DOX in the sample. UV was then employed to quantify the amount of free/unloaded DOX molecules in the combined supernatants. From the free DOX determined by UV, loaded DOX can be quantified based on the initial amount of DOX in the original solution. A calibration curve was obtained from a serial dilution of stock solution containing pure DOX in MilliQ water (concentration 1 mg/mL).

### Magnetic Experimental Set Up for MRs

Experiments were conducted on a Zeiss Axioplan 2 upright microscope using an Axiovert 503 mono camera and an Axiovert 200 inverted microscope with a Amscope MU903-65 camera. Experiments on the Axioplan microscope utilized a custom-built magnetic control system which applied magnetic field strengths at the sample from 3-7 mT. The magnetic system consists of four electromagnetic solenoids containing soft iron cores which are arranged in an array along the x and y axes, allowing for magnet fields to be applied in any orientation in the horizontal plane. The strength and direction of the fields are controlled using custom MATLAB code. Experiments performed on the Axiovert microscope utilized hand-held permanent cylindrical magnets that produced magnetic field strengths at the sample of approximately 10-30 mT. The hand-held permanent magnets were used when translating the cells due to their ability to be placed in closer proximity to the cells and, therefore, allowing for stronger magnetic field gradients to be applied compared to the electromagnets. Paramagnetic beads used in the experiments were fluorescent and had a diameter of 4.7 um. The fluorescent microspheres were illuminated using an X-Cite mini+LED source, as manufactured and designed by Excelitas Technologies Corp. of Salem, Massachusetts.

### Cell Culture and Maintenance

Human breast cancer cells (MCF-7) and Hepatocellular Carcinoma cells (HepG2) cells were provided by Richard West (Associate Scientist at Flow Cytometry Core Facility). Cells were cultured in Dulbecco's Modified Essential Medium (DMEM) and Ham's F-12 (Gibco, BenchStable, USA) media with 5% CO2 and maintained at 37° C. in an incubator. All experiments were performed after third passage of cells.

### Assessment of Cytotoxicity and Cellular Uptake of Microrobots

Cytotoxicity of MRs was evaluated in HepG2 cells. Cells were seeded (2×10⁴ cells/well) in a clear flat bottom 24-well plate (Corning™ Costar™ USA) and incubated in 1:1 mixture of Dulbecco's Modified Essential Medium (DMEM) and Ham's F-12 (Gibco, BenchStable, USA) media with 5% CO2 at 37° C. for 24 hrs. Then, cells were treated with MRs (4.7 μM size, 1 mg/mL) and incubated for 24 hrs. Cells were then imaged under optical microscope to check cell morphology. Cellular uptake of MRs was assessed as aforementioned method. HepG2 were treated with MRs (Carboxyfluorescein (green), 4.7 μM size) at a concentration of 100 g/mL and imaged after 24 hrs. Flow cytometry was also performed to quantify cellular uptake of MRs in HepG2 cells. 1×10⁵ cells/well were seeded in a 6-well plate and incubated in DMEM/F-12 with 5% CO2 at 37° C. for 24 hrs. Then, 20L of 1% w/v MRs solution was added to the each well containing 2 mL media. After 24 hrs, cells were washed and stained with CFDA-SE for 15 mins and analyzed using a flow cytometer, such as the BD FACSAria™ IIu flow cytometer, as manufactured and designed by BD Biosciences of Franklin Lakes, New Jersey. Comparative cytocompatibility and cellular uptake study of MRs with different surface functional group were carried out by same method.

### Movement of MRs Under the Influence of Magnetic Field

HepG2 cells were cultured (5×10⁵ cells/mL) in a 35 mm petridish at 37° C. with 5% CO2. After 24 hrs, MRs were added to the petridish and steered towards the cells using a wireless joystick or gaming controller. Images and videos were taken using an Axioplan 2 upright microscope using an Axiovert 503 mono camera and an Axiovert 200 inverted microscope with a Amscope MU903-65 camera.

### Characterization of DOX-MRs

Dry samples were used to record Fourier-transmission infra-red (FTIR) spectra to confirm successful conjugation of DOX to magnetic beads. Fluorescence images same DOX-MRs samples were taken using a fluorescence microscope, such as the Carl Zeiss Axiovert S100.

### Anticancer Effect of DOX-MRs

HepG2 cells were cultured (5×10⁵ cells/mL) in a 35 mm petridish at 37° C. with 5% CO2. After 24 hrs, DOXMRs were added to the petridish and incubated of 24 hrs. Cell death was assessed using fluorescence microscope. Another petridish was imaged for time-lapse video of DOX-MR uptake and cell death using a confocal microscope immediate after MRs addition.

### Assessment of Cell Death in 3D Spheroid

HepG2 cells were cultured in a petri-dish at 37° C. with 5% CO2 to generate three-dimensional (3D) spheroids to mimic tumor models in vitro. Spheroids were then treated with DOX-MRs for 24 hrs and cell death was evaluated using Tryphan blue assay, Spheroids were collected and washed with Dulbecco's phosphate-buffered saline (DPBS) before staining. 50 μL of 8% tryphan blue was added to 450 μL of media containing spheroids for 5mins and spheroids were imaged under microscope, such as the Carl Zeiss Axiovert S100.

### Cyto-Compatibility of Microrobots

Biocompatibility is one of the important factors while designing microrobots. Apart from being non-toxic, microrobots should not release any chemicals or by-products that affect results during experiments. In this example, the magnetic beads comprise polystyrene, which is considered nontoxic.

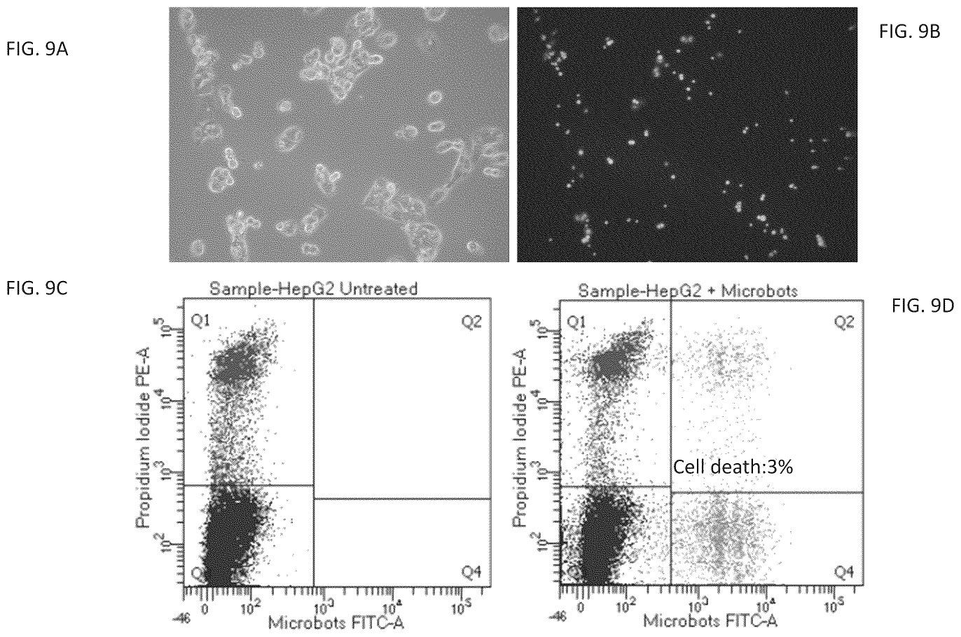

HepG2 cells were incubated with MRs for 24 hrs, and changes in cell morphology was monitored using an optical microscope. Cell morphology was found normal with intact clustered patterned of HepG2 cell growth (as shown in FIGS. 9A-9B). Flow cytometry data was in accordance with optical images with negligible (dead cells—3%) toxic effects (as shown in FIGS. 9C-9D). Nevertheless, cells were healthy even after 48 hrs of incubation. The data confirmed that magnetic beads were suitable for further modification and biological experiments.

### Cell-Internalization of Microrobots

Cellular uptake is an important consideration when cytotoxic drugs or drug delivery systems are developed or designed. Microrobots with different functional group were investigated based on their biocompatibility and cellular uptake. Neutral MRs showed highest cellular uptake as compared to carboxylic and amine functionalized groups in HepG2 cells. The mechanism of cellular uptake is presumably due to phagocytosis which includes two steps: adhesion followed by internalization. Polystyrene beads tend to adhere to the cell membrane, followed by internalization and this adhesion step is affected by anionic or cationic charges of the beads. Therefore, neutral MRs showed highest cellular uptake compared to other two charged MRS.

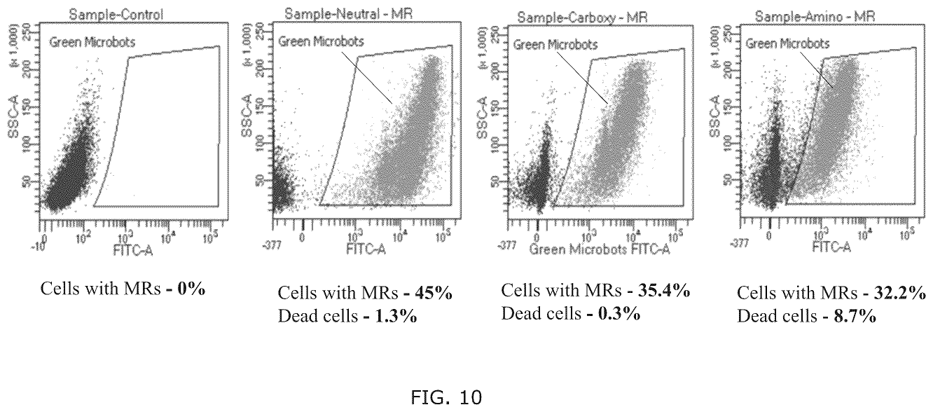

While neutral and carboxylic MRs were nontoxic, amine functionalized MRs showed slightly toxic effect with 17% cell death (as shown in FIG. 10). Interestingly, multiple MRs were observed inside the cells showing high affinity of these polystyrene beads towards the cells. Considering cytocompatibility, cellular uptake and functional group for further DOX conjugation, carboxylic MRs were selected for further experiments.

### Movement of MRs Under the Influence of Magnetic Field

Magnetic strength plays an important role in the movement of microrobots. The required field strength was investigated using a hand-held cylindrical magnet with magnetic field of 10-30 mT. Microrobots and HepG2 cells were aligned under the inventive magnetic controller with a wireless joystick. Direction and strength of the magnetic field was controlled in accordance with a custom MATLAB code.

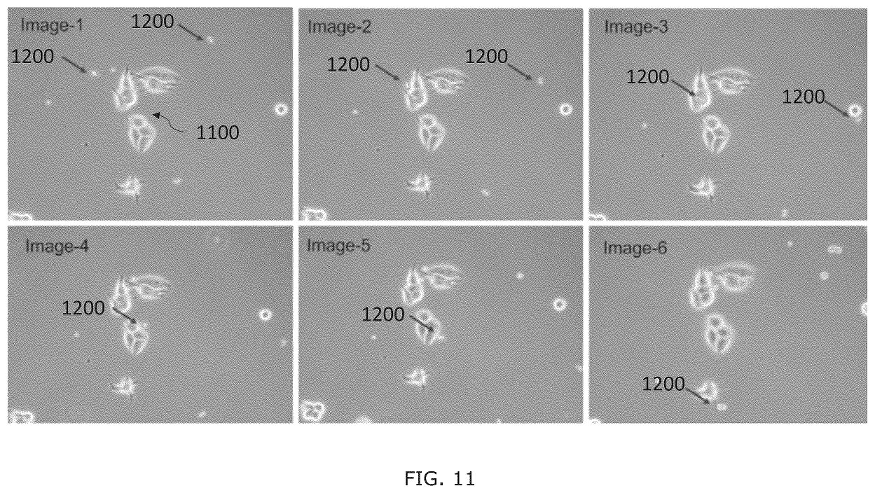

Microrobots were steered at any direction to bring them closer to the cells (as shown in FIG. 11). Besides that, MRs were also able to roll over or by-pass cells due to the precision in the inventive magnetic set up.

### Characterization of DOX-MRs

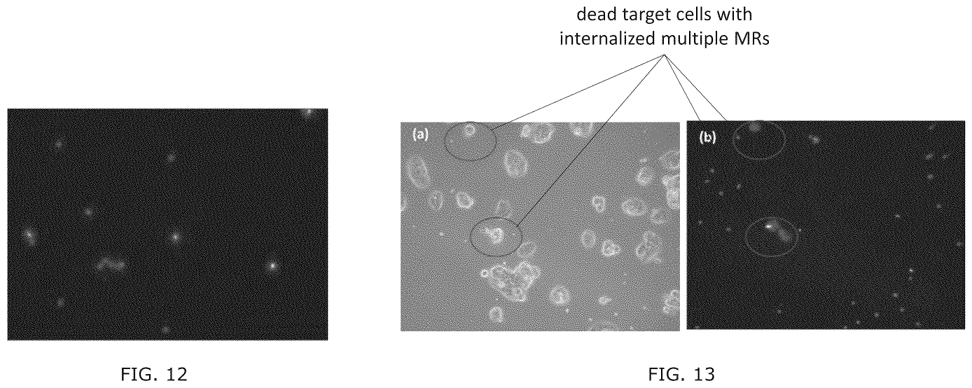

Chemical conjugation of DOX with microrobots were confirmed by Fourier transform infrared (FTIR) and fluorescence microscope. Additionally, intrinsic fluorescence of DOX was also used to confirm covalent linkage of DOX to MRs. Microrobots were imaged under a fluorescence microscope with Cy5.5 filter and bright red fluorescence from beads confirmed successful conjugation of DOX on the surface of microrobots (as shown in FIG. 12).

### In Vitro Anticancer Effect of DOX-MRs

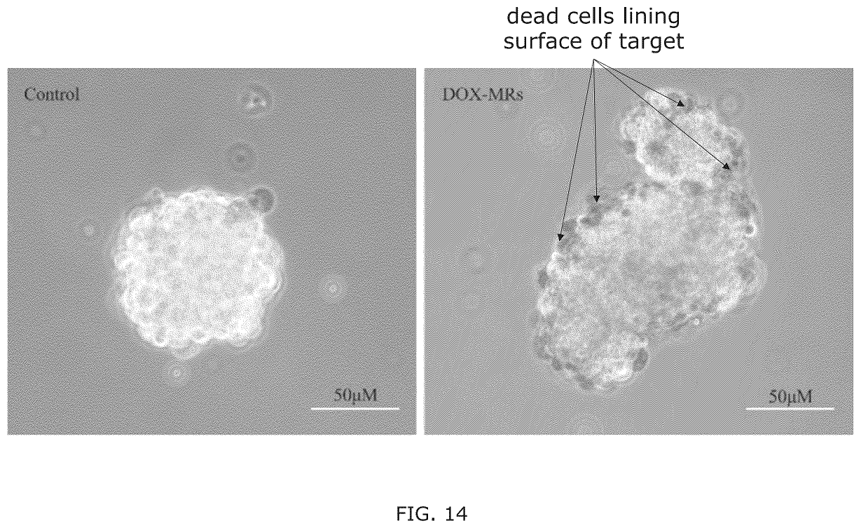

Doxorubicin (DOX) is an anthracycline which is known to damage DNA or interfere in DNA replication that eventually, leads to cell death. However, cytotoxic concentration of DOX has to be delivered to kill cells. HepG2 cells were treated with DOX-MRs and DOX-induced cell death was assessed after 24 hrs. Fluorescence images showed dead cells floating in the media with multiple MRs, whereas cells with single MRs were live (as shown in FIG. 13). This indicates that cell death was DOX concentration dependent and multiple MRs were able to deliver cytotoxic concentration of DOX to the cells. One advantage of the inventive method and controller is targeted delivery of therapeutic molecules at the target site without any invasive procedure. As confirmed by the results, cytotoxic concentration of DOX can be controlled by number of MRs being driven towards the cancer cell or tumor site. A time-lapse video also showed changes in cell morphology and induced cell death within 12hrs, assuming DOX release by action of protease enzymes was immediate after internalization. Moreover, anticancer effect was also evaluated in 3D spheroids as an in vitro tumor model. The results were inconsistent with 2D culture showing dead cells lining the surface of the spheroid (as shown in FIG. 14) after 24 hr incubation with DOX-MRs. This indicates that DOX-MRs can effectively kill cancer cells on the surface of the solid tumors and gradually decrease the size of the cancer cells, with repeated follow up treatments.

Therefore, this exemplary microrobotic delivery system of prepare drug-conjugated microrobots is a promising candidate for targeted chemotherapy. Other suitable applications of this microrobotic delivery system will be understood by one skilled in the art from the description herein, including conjugation of various biomolecules to microrobots and controlled delivery of required concentration thereof to the target site.

### Example 3

### Cellular Manipulation Using Rolling Microrobots

Magnetically driven microrobots provide the advantage of using a biocompatible driving mechanism with a large penetration depth. The inventive system utilizes rolling microrobots which can be rolled on surfaces and interfaces using a 3D electromagnetic coil setup (as shown in FIGS. 15, 3B) making them especially suitable for biological environments. Specifically, these rolling MRs are used in applications requiring maneuvering in blood vessels, such as removal of obstructions, or in cellular manipulation or delivery, such as cell sorting or biological research experiments as described herein and throughout the specification.

### Material and Methods

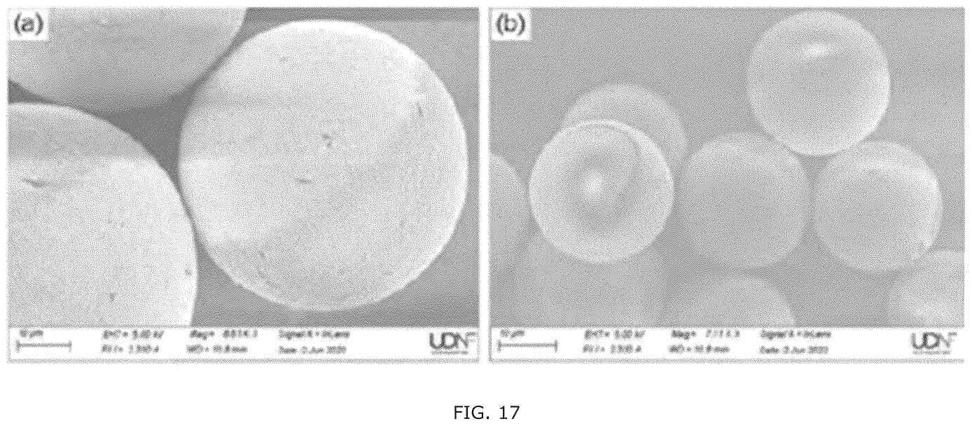

The microrobots were made using 45-85 μm diameter hollow silica spheres with a thin TiO₂ coating, as designed and manufactured by Cospheric of Goleta, California. The microrobots were also made using 20 μm diameter silica spheres with amine functionality, such as those designed and manufactured by Nanocs, Inc. of New York City, New York (Cat. No. Si20u-AM-1). The spheres were coated with nickel by e-beam deposition to make them magnetic (as shown in FIG. 16). Both the hollow and the solid silica spheres were coated with 100 nm of nickel; the deposition was performed at a 70-degree glancing angle in the case of the silica spheres (as shown in FIG. 16), which generally reduced the surface area that was coated. SEM images of the coated microrobots are illustrated in FIG. 17. Note that the magnetic moment of the spheres that have only a partial Ni coating tends to point in a direction tangent to the coated surface, whereas the spheres that are halfcoated in Ni produce a magnetic moment that points normal to the surface, which can be seen from the alignment of the microrobots when a magnetic field is applied.

### Mammalian Cells

Mammalian cells served as model cells for manipulation by the microrobots. Human breast cancer cells (MCF-7) and Hepatocellular Carcinoma cells (HepG2) cells were provided by Richard West (Associate Scientist at Flow Cytometry Core Facility). Cells were cultured in Dulbecco's Modified Essential Medium (DMEM, Gibco, BenchStable, USA) media with 5% CO2 and maintained at 37° C. in an incubator. All experiments were performed after third passage of cells. Cells were washed with Dulbecco's phosphate buffer (DPBS, Gibco, BenchStable, USA) and trypsinized to detach cells from the culture dish. All experiments were carried out with these single cells. The microrobots were mixed with cells and their movement and manipulation of the cells was recorded.

Experiments in which the cytocompatibility of the microrobots was measured were performed by mixing the microrobots with the cells prior to incubation. For the incubation with the cracked hollow silica microrobots, the microrobots were broken by using a pipette tip to push them against the side of the vial. Quantitative cell death was assessed by Trypan blue viability assay. The cells were collected in the media and after trypsinization. Then, the cells were centrifuged and suspended in phosphate buffered saline (PBS). The cells were then stained using 0.4% trypan blue and counted by a cell counter (Nexcelom Cellometer Vision Trio Cell Profiler). Cells (before trypsinization) were observed under a microscope for morphology analysis, followed by Trypan blue staining to quantitatively assess cell viability after 24 hours.

### Experimental Set Up

A 3D magnetic field system (as shown in FIGS. 15, 3B) was used to apply rotating magnetic fields in the xz, or yz planes for magnetic rolling. The system consists of four electromagnets arranged orthogonally in the xy plane and a pair of Helmholtz coils positioned beneath and above the viewing plane for applying z fields. Magnetic field strengths of approximately 5 mT were used in the experiments. The magnetic field strengths from each of the coils was controlled using custom matlab or python code which produced digital signals that were used to modulate the current sent to each of the coils. To apply rotating magnetic fields, discrete sinusoidal signals were sent to each coil, with a 90 degrees phase difference between the two pairs of orthogonal electromagnets that corresponded to the desired rotation axis. For example, to apply a rotating field in the xz plane, the x-axis current was set as Acos(2π f t) and the z-axis as Asin(2π f t), with A the magnitude and f the frequency.

Experiments were conducted on an Axiovert 200 inverted microscope with an Amscope MU903-65 camera. The microrobots were observed either on a glass slide or within an enclosed chamber (Grace Bio-Labs SecureSeal Hybridization Chamber).

### Results

The microrobots were rolled by applying rotating magnetic fields either in the xz or yz planes, which resulted in the translation of the microrobots in the y or x directions, respectively. Microrobots were moved both on the solid substrate as well as at the air-liquid interface (as shown in FIG. 18). The direction of motion at the air-liquid interface is the same as that on the solid surface for a given rotating field orientation, which can be explained by the larger drag on the bottom of the microrobot compared to that at the top in both cases. Buoyant microrobots were also rolled on the top surface of a sealed chamber, which results in the microrobot translating in the opposite direction. The speed of the microrobots could be tuned by varying the magnetic field rotation frequency, v. As shown in FIG. 18, the measured speed of the solid silica and hollow microrobots (both on the glass substrate and at the air-liquid interface) as a function of rotating magnetic field frequency. As can be seen from the plot, the microrobot speed increased with frequency approximately linearly, as expected for a rolling object in which the speed is approximately equal to 2πRf, with R being the microrobot radius and f the rotation frequency. Note that, due to slip, the actual speed of a rolling microrobot in a fluid is reduced. As illustrated in FIG. 17, one can see that the speed of the microrobots is considerably less than 2πRf, signifying that slip is significant. Notably, the hollow spheres moved faster at a given frequency at the air-liquid interface than on the solid substrate, indicating that they experienced less slip at the interface.

Above a threshold frequency, the microrobot was unable to maintain synchronicity with the magnetic field and its speed decreased. The threshold frequency at which this occurs depends on the relative degree of magnetic torque compared to the amount of rotational drag. This threshold frequency was found to be dependent on the size of the microrobot and the amount of nickel coating on its surface, but generally occurred above about 10 Hz.

The buoyant hollow microrobots were able to move at speeds of up to about 300 μm/s at the air-liquid interface and about 125 μm/s on the glass substrate, while the smaller silica microrobots attained speeds of around 75 μm/s on the substrate (as shown in FIG. 20). Due to the adhesive and physical interaction with the cells, the maximum microrobot speeds were typically reduced in environments containing cells.

At relatively high densities, the microrobots sometimes formed aggregates due to attractive magnetic dipole interactions. For example, chains and clumps of microrobots tumbled end over end when a rotating field is applied. The chains tend to tumble rather than roll, due to their magnetic moment residing along their long axis and therefore aligning with the applied magnetic field. This behavior is similar to that previously observed for chains and clusters of paramagnetic spheres.

Hollow microrobots would sometimes crack, resulting in them sinking to the bottom substrate. These microrobots were used to manipulate cells by rolling them to the cells and either pushing the cells or, in some cases, carrying the cells (as shown in FIG. 19). It was found that single microrobots generally could push but usually could not carry the cells, while larger clumps of microrobots were more likely to “pick up” cells by rolling them into the cells and carrying the cells with them. Although both microrobots tended to adhere to the cells, the hollow microrobots were more effective at carrying the cells, possibly due to their larger size or different surface properties. The origin of the adhesion between the cells and the microrobots may be due to an electrostatic attractive force or Van der Waals attraction. It was also found that spinning the large hollow microrobots at high frequency in the xy plane sometimes resulted in the cells detaching from the microrobots.

Microrobots that were rolled at high frequencies created flows in the fluid which resulted in the advective motion of the cells. These flows tend to carry cells that were behind the microrobot forward while pushing those in front of it farther away, similar to what one would expect from the flow field produced by a neutral squirmer. Such manipulation using fluid flow created by a rolling microrobot is similar to previous work in which a rotating rod created a vortex that trapped protein crystals near the rod, allowing them to be moved with the rod. Thus, the flow fields created by a hollow microrobot rolling at high frequency caused the cells in near proximity to the microrobot to move with the microrobot.

### Example 4

### Cytocompatibility

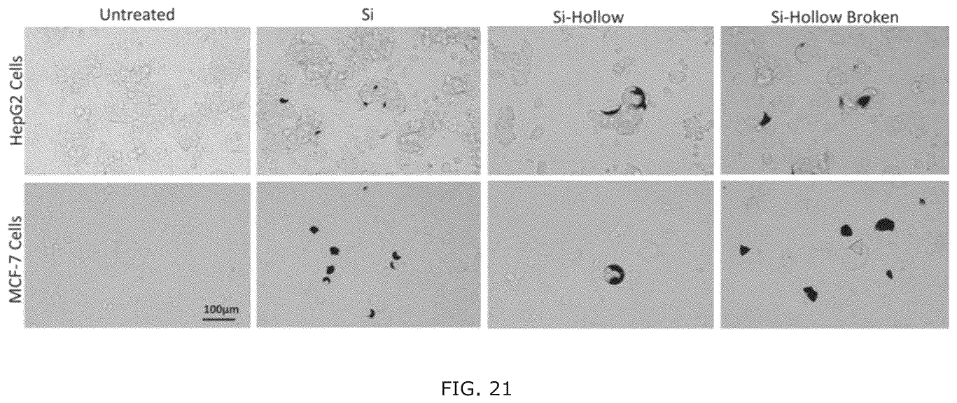

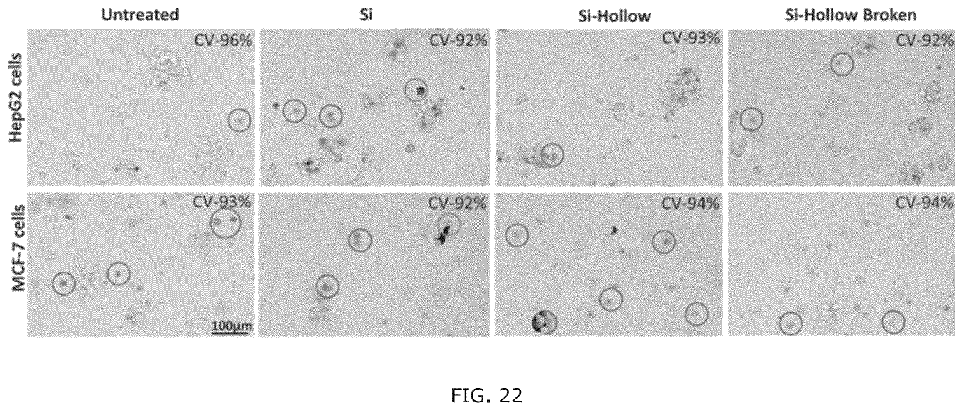

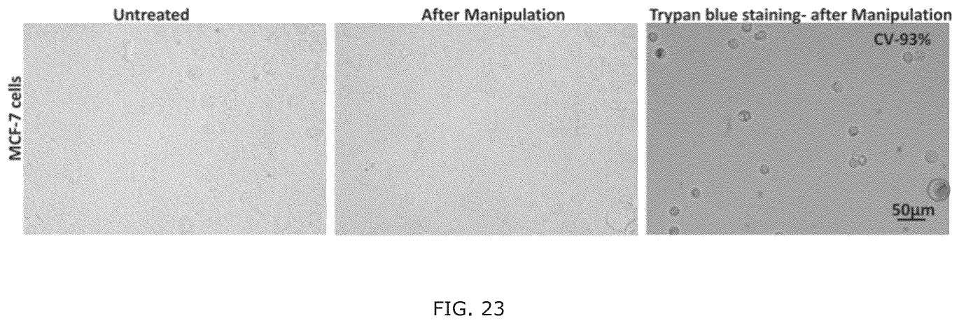

The cytocompatibility of the microrobots with the cells was also assessed. The cytocompatibility of the rolling microrobots was assessed in both HepG2 and MCF-7 cells. Cells were incubated for 24 hours with silica, hollow TiO2 coated silica, as well as cracked hollow TiO2 coated silica microrobots that were broken before adding them to the culture medium. FIG. 21 shows brightfield images of the cells after incubation along with a control in which no microrobots were added. As can be seen in the images, cells preferentially attached to the microrobots, therefore providing another method for loading the cells onto the microrobots for transport and delivery. The cell morphology and growth patterns were intact for both types of cells, indicating that the microrobots are not toxic to the cells. Trypan blue staining was also performed to quantitatively assess cell viability. The results show that the microrobots are not toxic to the cells (as shown in FIG. 22). The viability of the cells was also tested after manipulation by the microrobots. As shown in FIG. 23, which depicts images of MCF-7 cells before and after manipulation, as well as Trypan blue staining results, the cells are still viable after manipulation. Thus, manipulation of cells by means of magnetically rolling microrobots, which shows promise as a robust means of motility in biological applications, such as in blood vessels. Aside from manipulation, the microrobots can also be effectively navigated in an environment populated with cells, which is relevant to many biological systems. This technique therefore allows for quick microscale assembly and manipulation with high precision.

### Example 5

### ModMag: A Modular Magnetic Micro-robotic Manipulation Device

Electromagnets generate magnetic fields by moving current through a conductor. Electromagnetic actuation has the ability to actuate magnetic objects untethered, i.e. without touching them. This is particularly useful in the field of micro-robotics, in which the small size of the micro-robots makes tethered actuation impractical. Micro-robots have drawn a lot of attention in recent years due to their potential to improve the efficacy of various tasks, such as cell manipulation, targeted drug delivery, microsurgery, and mixing of particles.

One method of controlling micro-robots is magnetic actuation. Magnetic actuation desirably permit untethered actuation capability, making it possible to deploy the micro-robots in a variety of environments. In addition, magnetic fields are regarded as a safe choice to use at the cellular and tissue level for a large number of biomedical applications, and can generate relatively large forces. Conventional electromagnetic actuation mechanisms for micro-robotic control typically use expensive equipment (e.g. data acquisitions devices and large bench top power supplies) that limit their ability to be used on other microscopes or transported to other locations. Furthermore, the software is generally integrated onto a lab computer, which limits portability.

### Electromagnetism Principles

When an electric current passes through a wire, the movement of charge generates a magnetic field. A solenoid, or electromagnet, uses many of these wires wrapped concentrically around a cylinder to create a magnetic field in a direction along its central axis. The strength of the magnetic field is proportional to the current and increases with the number of times the wire is wrapped around the cylinder. It is also proportional to the magnetic permeability which can be increased by introducing materials with high magnetic permeability into the core of the solenoid. Generally, ferromagnetic materials with low coercivity, such as soft iron, enhance the magnetic field generated by the solenoid while retaining low magnetization when the current is turned off.

Ferromagnetic materials contain microscopic magnetic domains where groups of magnetic moments naturally align in the same direction. When the domains are all aligned randomly the material is demagnetized. However, the presence of an external magnetic field can align the domains in the direction of the applied field, causing the material to be magnetized. Thus, when current passes through the surrounding coil of wire, it forces each of the magnetic domains in the same direction, thereby amplifying the field. In a similar manner, when a ferromagnetic object, such as a magnetic micro-robot, is placed in a magnetic field, its magnetic moment aligns with the external field. In one example, this allows for the magnetic steering of a self-propelled, spherical, iron coated micro-sphere suspended in a liquid solution.

Typically, the micro-sphere moves in an arbitrary direction and is subject to Brownian rotational fluctuations. An external torque can be applied in order to steer the microsphere in a particular direction. The torque, F, is given by the expression: r=m×B, where m is the magnetic moment of the micro-robot and B is the magnetic field. This torque therefore allows for magnetically orienting the micro-robots.

Magnetic forces can also be applied to the micro-robots by applying magnetic gradients. The magnetic force, F, is given by the following expressions: F=(m·V) B. Applying sufficiently strong magnetic gradients generally requires the use of high permeability poles which are used to concentrate the magnetic flux generated from an electromagnetic. This allows for strong enough magnetic gradient forces to overcome the highly viscous drag forces that a micro-robot experiences. Magnetic devices that are used to magnetically manipulate objects are known as magnetic tweezers. In one example, an object with a magnetic moment can be oriented or translated in a uniform or spatially varying field.

### Experimental Set Up

### (A) 2D Traditional Coil System

The traditional 2D electromagnetic setup consists of a 3D printed rectangular exchangeable stand that fits inside the microscope slide holder, and 4 perpendicular electromagnets (see FIGS. 24A-24B). The stand was designed such that it fit onto the mechanical stage of a microscope, such as a Zeiss Axioplan 2 upright microscope. However, one skilled in the art would understand from the description herein that the invention is not limited to a particular microscope stage surface. The Axioplan 2's limited stage space of approximately 150×100×25 cm in volume made it desirable for the stand and the electromagnetic coils to be as compact as possible.

The 2D electromagnetic stand was designed in Solidworks and consists of only one part (see FIGS. 24A-24B). The height of the stand measures roughly 23 mm to accommodate for the rotation of all microscope objectives in the objective turret. A standard 22×22 mm cover-slip or 25×25 mm square glass slide could be loaded with the desired samples up to the height of the stand. In another example, a 30 mm petri dish can be fitted in the region of interest, thereby allowing the electromagnets to point directly towards the samples. Additionally, or optionally, a custom square-shaped holder can render the use of a microscope slide moot and the electromagnets would rest at an angle. Still further, a slit in the stand is provided to allow for easy insertion and removal of the cover slip using laboratory tweezers.

The 2D electromagnetic stand was printed via additive manufacturing using a suitable machine, such as a Comgrow Creality Ender 3 Pro 3D printer using 1.75 mm, white polylactic acid (PLA) filament. An digital file format, such as a Standard Triangle Language (STL) file, of the stand was uploaded to Creality's Cura software with the following settings: printing temperature: 200° C., bed temperature 60° C., speed: 50 mm/s, layer height: 0.12 mm, and infill: 20. The rest of the settings were left at default. These settings provide a strong and durable stand for the electromagnets to rest upon. Four equally spaced holes 5 mm in diameter were created along the same plane as the cover slip holder allowing for the insertion of each electromagnet core. The holes allow for the electromagnetic core to be as close to the cover slip as possible.

The electromagnetic core material is AISI 1008 carbon steel made of 99.31-99.7% iron, cold drawn and annealed at 925° C. The cores had raw dimensions of 5 mm in diameter by 200 mm long. The relative permeability of the core was listed as (86±3.61) H/m. The cores were then cut to 50 mm long. 24 AWG magnet wire was used for the electromagnetic coils. In order to prevent the coil from unraveling, plastic end caps of 30 mm in diameter were cut from 0.7 mm in thickness plastic. 5 mm holes were then drilled into the center of these circles to allow for the electromagnetic core to be inserted.

To create an electromagnet, one end cap is positioned at the end of the core and glued in place using Gorilla 2 part epoxy. The second end cap is then glued 50 mm from the first end cap, or 5 mm from the opposite side. As a result, the electromagnetic coil is 50 mm long, leaving 5 mm at one side to be inserted into the stand. Coils were then wound carefully using an electric drill to exactly 980 turns of the 24 AWG magnet wire. Using a Tesla meter, the electromagnetic strength at the face of the coil was measured to be (201±3) mT at a current of 2 Amps, and around 15 mT at the center of the work space. The six electromagnet's were then inserted and glued into the six holes in the 3D printed stand.

### (B) 3D Helmholtz System

A 3D electromagnetic manipulation system is suitable for micro-robotic applications involving rotating homogeneous magnetic fields for facilitating 3D magnetic steering or magnetic rolling. The custom coil system is mounted on a microscope, such as the Zeiss Axiovert 200 M inverted microscope (see FIGS. 25A-25B).

In one application, measurements of the microscope bed dimensions and distances between lenses were recorded. These size constraints defined the maximum radii for the coil rings. Calipers were used to determine the microscope measurements. The Helmholtz coil system consists of three pairs of rings each sharing their respective common axis. The rings are wrapped with copper wire to form coils. In one example, to attain an optimally uniform field, the distance between pairs of rings should be equal to their radius.

To optimally fit in the workspace, the relatively larger sized rings were mounted on the stage vertically while the relatively smaller rings were mounted horizontally, as shown in FIG. 25. In one example, microscope dimensions allow for a maximum diameter of 106 mm for the larger rings without interfering with the objective turret, and a minimum diameter of 36 mm for the smaller rings without interfering with the condenser lens. The smaller or smallest pair of rings are spaced such that their distance, Hs, is equal to their radius, Hs=Rs=26 mm (both measured center-to-center). The medium sized pair has a radius of Rm=35 mm and is spaced slightly farther apart (Hm=66mm). For the larger or largest pair of rings, RI=54 mm and HI=84 mm. As a result, the ratios of distance over radius of the small, medium, and large pairs of rings are 1, 1.9, and 1.6, respectively.

The platform unit is roughly 230 mm long and 140 mm wide with integrated slots for mounting on the microscope. On either side of the platform is an area for mounting a motherboard with electrical connections, and a square divot for mounting a 2-axis micrometer for slide arm micro-translation. The slide arm fits both a conventional glass microscope slide as well as square cover-slips. Four slots are outfitted within the platform to hold the large and medium coils with a transition fit. The small coil pair sits on four stilts, while four small pegs separate the coil pair.

The platform, slide arm, and rings were designed within Solidworks CAD software and printed using additive manufacturing or 3D Printing methods. All components are printed with PLA using an Ender 3 Pro. Structural properties of PLA allow for adequate rigidity within the frame to hold the weight of the primary components. The coils are wrapped with 24 AWG copper wire using a standard hand drill to achieve more uniform wire layers than what is achieved by hand. In order to connect the ring to the drill, a drill adapter unit consisting of three extending limbs was fabricated. Small pegs on each limb allowed for a transition fit into receiving holes on the rings. The resulting number of turns is approximately 368 for the small and medium coils and 260 for the large coils. Applying 1 A to each of the coils yields a uniform magnetic field over the sample area of 4 mT, 2 mT and 2 mT for the small, medium and large rings respectively. This allows for magnetic steering and rolling actuations of magnetic micro-robots, as further discussed below.

### (C) Magnetic Tweezer System

The 2D magnetic tweezers system for control of magnetic micro-robots utilizes a quadrople design and illustrated in FIGS. 26A-26B. Its main components are divided into three parts, namely the yoke, poles, and coils. In one example, the yoke was designed using SolidWorks and subsequently fabricated using Protopasta Magnetic Iron Filled PLA through 3D printing. The yoke possesses an outer diameter of 180 mm and an inner diameter of 160 mm. The use of magnetic PLA provides several advantages, including completing the magnetic circuit to increase the efficiency of magnetic field generation, introducing high magnetic permeability to reinforce the magnetic field, and reducing the current required to decrease the heat generated by the coils.

Four magnetic poles are integrated into the yoke, with sharp tips oriented toward the center. Each pole is positioned on the same level and spaced 90 degrees apart from one another. The distance between opposite tips is 3 mm. These poles are made using 1.5 mm Nickel-Iron soft ferromagnetic alloy (Mu-Metal, Magnetic Shield Corporation), a high-permeability material fabricated using laser cutting. This material allows efficient transport of magnetic flux from the coils to the tip.

Each magnetic pole has an electromagnetic coil fixed to it, comprising 600 turns of 24 AWG enameled Copper wire. The tip of the magnetic pole generates a strong magnetic field gradient when the electromagnetic coil receives an input current, which then generates a magnetic force acting on the micro-robot. The measured magnetic field directly in front of a single pole was measured to be 120 mT at 2 A. Approximately 3 mm away from this pole, the field was measured to be approximately 40 mT. This results in a magnetic gradient of roughly 26 T/m. By applying different currents to different coils, the magnitude and direction of the micro-robot's motion can be changed based on the principle of force superposition.

As with the other systems described above, space availability constraints are determined by at least the dimensions and configurations of the microscope. Accordingly, the invention can be modified based on specific space constraints of system components, such as the microscope.

### Electronics or Electrical System

The electrical system is designed to work with any of the electromagnetic setups discussed above (or variations thereof). It is also capable of standalone operation without being tethered to a single lab computer, thereby providing portability. Portability means the system can be easily transported without the need for a bench-top external power supply.

Computational capability is provided by a processor, such as Raspberry Pi Model 3 B+. To facilitate user interaction, a custom graphical user interface has been developed in Python and is displayed on a HMTECH 10.1 Inch Touchscreen Monitor with a resolution of 1024×600 (as shown in FIGS. 27B, 28) The computer is also equipped with 40 GPIO (general purpose input/output) pins, which can be controlled through software. However, as electromagnets require high currents that cannot be powered directly from the GPIO pins, six SEEU. AGAIN BTS 7960 43A Double DC Stepper H-Bridge PWM drivers are employed to vary the current and polarity from an external power supply. These H-Bridges convert the user-defined currents generated from the GPIO pins and scale them accordingly to each electromagnet.

The external power supply comprises three Lithium-ion 18650 batteries, providing 12V with a peak current draw of 3 A. To facilitate charging, a charging circuit board with a DC barrel jack has been incorporated, allowing the batteries to be recharged when depleted. External power sources may also be used if desired. Connection between the controller and each of the three coil setups is facilitated through a 20-pin IDC cable with an associated female adapter terminal. The electrical schematic is presented in FIG. 27A.

A HiLetgo DDS AD9850 Signal Generator Module is provided to produce the signals required for actuating acoustic micro-robots. To achieve this, the signal generator chip is connected to a piezoelectric transducer that is mounted on a microscope slide. When activated, the signal generator produces an electrical sine wave that is converted into pressure waves at the same frequency by the transducer. These pressure waves travel through the liquid medium on the slide's surface until they reach the acoustic micro-robot. Once there, they vibrate a small air bubble that is trapped inside a cavity. This vibration generates an acoustic streaming force that propels the micro-robot forward. Typically, the signal generator module is a large bench-top device, given the high actuation frequencies involved (1-3 MHz). In contrast, the inventive electrical system integrate all the necessary features for actuating acoustic micro-robots into a single chip on the device, making it highly compact and portable.

### Software

To achieve precise real-time control of the current to each electromagnetic coil, a custom graphical user interface was developed in Python using the Tkinter and Gpiozero libraries. This intuitive software allows for the application of constant magnetic fields in any user-defined direction, as well as rolling, spinning, or vibrating functionalities. Additionally, the system can be controlled using a game controller to achieve even greater responsiveness and precision.

To apply current and therefore produce a magnetic field, the user inputs the desired magnetic field direction and strength, which is then used to modulate the current sent to each coil. This involves breaking down the associated vector into its x and y components, and scaling the current sent through each coil proportionally to the magnetic field strength. Furthermore, to improve the field strength and uniformity, an opposite polarity signal is sent to the opposite-facing coil. For example, when a positive current is applied to the-x electromagnet coil, a simultaneous negative current will be applied to the +x coil, enhancing the magnetic field strength and uniformity.

A magnetic tweezer mode allows the user to switch between “tweezer on” and “tweezer off” depending on the type of electromagnetic system being used. Magnetic tweezers utilize traditional coils to concentrate magnetic flux towards the poles, creating sufficient magnetic gradients to move a magnetic micro-robot without additional propulsion mechanisms. To optimize micro-robot movement, the magnetic moment should align with the magnetic gradient. Thus, when magnetic tweezer mode is enabled in the software, current is only sent through one pair of coils in order to align the magnetic moment with the field gradient. The polarity of the magnetic field is not as important in this case since a single pole always acts to attract the micro-robots. This is because changing the polarity changes the sign of B (external magnetic field) and this also causes the micro-robot to rotate such that m (magnetic moment) aligns with B, hence changing the sign of m as well.

In addition to being controlled via the graphical user interface, all functions can be operated using a gaming controller, as illustrated in FIG. 28. When a button is pressed on the interface, it wirelessly connects to a USB gaming controller. In one example, the left joystick controls the orientation or tweezer functionalities in 360° while the right joystick adjusts the direction of a rolling magnetic micro-robot, controlling the rotating magnetic field.

In another example, a quick magnetic field gradient is generated by using the 4 direction D-Pad on the controller. With the device, a rotating magnetic field can be generated in any user-defined direction, enabling precise and responsive control of a rolling micro-robot or an artificial bacteria flagella micro-robot/helical micro-robot. This is achieved by applying a time-varying sine wave to each of the X, Y, and Z axis coils, using the equations below:

$$B x = A [ cos ( γ ) cos ( a ) cos ( ω t ) + sin ( a ) sin ( ω t ) ] Bz = A sin ( γ ) cos ( ω t ) \qquad ( 3 )$$

$$By = - A [ cos ( γ ) sin ( a ) cos ( ω t ) + ( cos ( a ) sin ( ω t ) ] \qquad ( 4 )$$

$$Bz = A sin ( γ ) cos ( ω t )$$

In the expression above, y is the azimuthal angle from the Z axis, a is the polar angle from the X axis, A is magnetic field magnitude and ω is the frequency which controls the speed of the rolling micro-robot. The default azimuthal angle for controlling magnetic rolling micro-robots is 90°, whereas changing the polar angle allows the user to steer the micro-robot's direction. In contrast, changing both the azimuthal and polar angles provides full 3D maneuverability for helical micro-robot swimmers. The Y button feature allows the user to quickly switch between rolling and spinning actuation methods, which can also be achieved through the GUI by varying the value of gamma to 0° for clockwise spinning or 180° for counterclockwise spinning.

Additionally, the graphical user interface includes options to control the micro-robot. In one example, the ability to magnetically vibrate the micro-robot is provided by turning on and off opposite facing electromagnets at user-defined frequencies. Moreover, there are quick direction buttons available that enable the user to apply magnetic fields in the ±x or ±y directions without having to use the slider to select specific angles. Furthermore, the device employs a DDS signal generator module to control the frequency of a piezoelectric transducer and therefore actuate an acoustic micro-robot. The user can adjust the frequency to actuate a range of acoustic micro-robots, and to turn on the module, the user can either use a button on the GUI or press the A button on the gaming controller. When the module is activated, the acoustic micro-robots self-propel, and the direction and orientation of the micro-robot can be adjusted using the magnetic field controller.

A touchscreen display may be provided to facilitate the above-discussed control options, or may be wirelessly facilitated via the PIGPIO daemon, which not only enables the GUI Python program to be run and controlled on any computer on the same Wi-Fi network but also allows for the integration of the magnetic control system with other software on the computer. For instance, the magnetic software can be used for real-time feedback control while tracking micro-robots using Python's OpenCV library and TrackPy.

### Results or Experimental Validation

### (A) 2D Traditional Coil System

The 2D traditional coil setup can orient a variety of self-propelled micro-robots, such as electrophoretic or diffusiophoretic magnetic Janus microspheres, or bubble-propelled micro-robots. To investigate the dynamics and control of platinum and nickel coated bubble-propelled micro-robots, the inventive 2D traditional coil setup and controller was configured to manipulate passive polystyrene particles into the letters U and D, illustrating the versatility of the system. FIG. 29 displays the trajectory of a 25 μm bubble-propelled micro-robot on the air-water interface, whose orientation or direction is regulated using the inventive magnetic controller.

As illustrated in FIG. 29, the trajectory of self bubble propelled magnetic microrobot on the air-water interface was observed over approximately 13 seconds. The microrobot is a 25 um polystyrene microspheres coated in 100 nm Ni and 25 nm Pt. The fluctuations from a perfectly linear trajectory are due to subtle air currents moving the robot around. Scale-bar: 200 um.

### (B) 3D Helmholtz System

The 3D Helmholtz system can control various types of magnetic rolling micro-robots, such as micro-spheres coated with a hemisphere of nickel through electron beam deposition. In one example, the 3D Helmholtz system is employed in to showcase the ability to manipulate cells using rolling magnetic micro-robots. Moreover, the rotating magnetic field produced by the controller enables the rolling of Cell-bots, which are biological cells with magnetic micro-robots ingested inside. An illustration of this application is depicted in FIG. 30. The system has also been employed to actuate magnetic helical micro-robotic swimmers.

As shown in FIG. 30, the trajectory of a synthetically engineered Chinese hamster ovarian cell is manipulated by ingested magnetic microrobots. The micro-robots used were 4.8 um silica micro-beads coated with 100 nm of Nickel. Scale-bar=20 um.

As shown in FIG. 31, the combination of the DDS module and rotating magnetic field capabilities allow for steering acoustic micro-swimmers. The left joystick of the gaming controller is used to orient the micro swimmers while toggling the acoustic signal. The system also allows for rolling of the acoustic micro-swimmers with the acoustic signal on or off, providing more refined and flexible control options.

### (C) Magnetic Tweezers System

The magnetic tweezers system utilizes high magnetic field gradients to direct magnetic micro-beads and is applicable in cases where self-propulsion is not feasible or when high forces are required. FIG. 32 illustrates the trajectories of two 25 μm polystyrene microspheres coated in Nickel and moving through water on a microscope slide. The microspheres were actuated using the magnetic tweezers and the inventive device.

Although described herein in an experimental proof-of-concept arrangement in which the micro-robots to be manipulated are disposed in a petri dish and imaged using microscopic imaging devices using visible light radiation, it should be understood that the invention is not limited thereto. Embodiments of the control systems may be implemented to control a micro-robot disposed within a portion of a patient's body (preferably immovably stabilized) and visualized in real time using imaging devices having microscopic precision and using non-visible wavelengths to help non-invasively directing a robot inside the patient from outside the patient. The patient may include any type of animal, but ideally may include humans or other mammals.

Although the invention is illustrated and described herein with reference to specific embodiments, the invention is not intended to be limited to the details shown. Rather, various modifications may be made in the details within the scope and range of equivalents of the claims and without departing from the invention.

1. A non-invasive method of manipulating at least one target cell, the method comprising:
treating the at least one target cell with one or more biocompatible magnetic microrobots (MRs); and
subjecting the one or more MRs to a magnetic oscillation profile via an external magnetic control assembly; and
causing the magnetic MRs to move or vibrate in response to the external magnetic control assembly and causing the at least one target cell to move or vibrate in response to motion or vibration of the one or more MRs.

2. The method of claim 1, wherein the magnetic control assembly comprises a plurality of electromagnetic solenoids arranged in an array, and a pair of electromagnets comprising a first electromagnet and a second electromagnet opposite the first electromagnet.

3. The method of claim 2, wherein the magnetic oscillation profile comprises one or more of a magnetic field orientation and a magnetic field strength.

4. (canceled)

5. The method of claim 3, wherein subjecting the one or more MRs to a magnetic oscillation profile comprises applying an oscillating square wave of predefined frequency range for a predetermined duration to the pair of electromagnets.

6. (canceled)

7. (canceled)

8. (canceled)

9. The method of claim 1, further comprising treating the one or more MRs for conjugation with a therapeutic biomolecule.

10. (canceled)

11. (canceled)

12. The method of claim 1, further comprising delivering a predefined concentration of the therapeutic biomolecule to the at least one target cell, via the one or more MRs when the one or more MRs is internalized by the at least one target cell.

13. The method of claim 1, wherein the at least one target cell is a cancer cell, and delivering the predefined concentration of the therapeutic biomolecule to the at least one target cell causes apoptosis of the at least one target cell or a decrease in size of the at least one target cell with the predefined concentration of the therapeutic biomolecule relative to at least one target cell without the predefined concentration of the therapeutic biomolecule.

14. The method of claim 1, wherein the one or more MRs comprises hollow silica spheres and solid silica spheres, and the method further comprises depositing a full or partial metal coating on the one or more MRs to produce a magnetic moment.

15.-20. (canceled)

21. The method of claim 1, wherein the external magnetic control assembly is connected to a portable control panel configured to control one or more parameters of the magnetic oscillation profile.

22. The method of claim 1, further comprising a portable gaming controller connected to the control panel, and the one or more parameters include an input related to a characteristic of a motion of the one or more MRs.

23. A non-invasive control system for manipulating at least one target cell, the system comprising:
one or more biocompatible magnetic microrobots (MRs);
an external magnetic control assembly for subjecting the one or more MRs to a magnetic oscillation profile; and
a control device connected to the external magnetic control assembly, the control device configured to control one or more parameters of the magnetic oscillation profile;
wherein the magnetic MRs are configured to move or vibrate in response to the external magnetic control assembly and to cause the at least one target cell to move or vibrate in response to motion or vibration of the one or more MRs.

24. The system of claim 23, wherein the one or more parameters of the magnetic oscillation profile comprises one or more of a magnetic field orientation and a magnetic field strength.

25. The system of claim 24, wherein the magnetic control assembly comprises a plurality of electromagnetic solenoids arranged in an array, and a pair of electromagnets comprising a first electromagnet and a second electromagnet opposite the first electromagnet.

26. (canceled)

27. (canceled)

28. The system of claim 24, wherein the magnetic control assembly comprises a pair of permanent magnets positionable adjacent the one or more MRs.

29. (canceled)

30. (canceled)

31. (canceled)

32. The system of claim 24, wherein the magnetic control assembly comprises a plurality of electromagnets arranged orthogonally along an x-y plane and a pair of Helmholtz coils positioned opposite of each other for a distance.

33. (canceled)

34. (canceled)

35. (canceled)

36. The system of claim 23, wherein the control device comprises a control panel and a processor coupled to the control panel and the magnetic control assembly, wherein the processor is configured to transmit an input relating to one or more parameters of the magnetic oscillation profile of the magnetic control assembly.

37. The system of claim 23, wherein the control device comprises a portable gaming controller connected to the control panel via a communication interface, and the portable gaming controller is configured to provide an input related to a characteristic of a motion of the one or more MRs.

38. The system of claim 24, further comprising a microscopic imaging device configured to enable imaging of the at least one target cell, the one or more MRs, or a combination thereof.

39. The system of claim 38, wherein the magnetic control assembly comprises at least one pair of electromagnets arranged perpendicular to each other.

40.-50. (canceled)

51. The system of claim 38, wherein the magnetic control assembly comprises a yoke having a plurality of magnetic poles, each one of the plurality of magnetic poles having an electromagnetic coil and a tip that is spaced apart from each other and oriented radially inward.

52. (canceled)

53. (canceled)

54. The system of claim 36, wherein the control device comprises drivers configured to vary a current and a polarity applied to the magnetic control assembly by an external power supply.

55. (canceled)

56. (canceled)

57. (canceled)

58. The system of claim 36, wherein the magnetic oscillation profile comprises modulation of a current applied to the magnetic control assembly.

59. (canceled)

60. The system of claim 36, wherein the control device includes a magnetic tweezer configured to create magnetic gradients for moving or vibrating the MRs.

61. (canceled)

62. A non-invasive method of manipulating at least one target cell, the method comprising:
treating the at least one target cell with the non-invasive control system of claim 22; and
subjecting the one or more biocompatible magnetic microrobots (MRs) to the magnetic oscillation profile via the external magnetic control assembly and the control device connected to the external magnetic control assembly and configured to control one or more parameters of the magnetic oscillation profile; and
causing the magnetic MRs to move or vibrate in response to the external magnetic control assembly and causing the at least one target cell to move or vibrate in response to motion or vibration of the one or more MRs.
